(19) United States
(12) Patent Application Publication
(10) Pub. No.: US 2024/0368555 A1
(43) Pub. Date: Nov. 7, 2024

(54) CELLS HAVING ABILITY TO INDUCE SKIN APPENDAGES, AND PRODUCTION METHOD THEREOF

(71) Applicant: Masakazu Kurita, Tokyo (JP)

(72) Inventor: Masakazu Kurita, Tokyo (JP)

(21) Appl. No.: 18/562,467

(22) PCT Filed: Mar. 30, 2022
(86) PCT No.: PCT/JP2022/015867

§ 371 Date: Nov. 20, 2023

(30) Foreign Application Priority Data
May 21, 2021 (JP) 2021-085915

(51) Int. Cl. C12N 5/0775; A01K 67/0271; C07K 14/435; C12N 15/64; C12N 15/86

(52) U.S. Cl. CPC C12N 5/0668; A01K 67/0271; C07K 14/435; C12N 15/64; C12N 15/86; A01K 2227/105; A01K 2267/03; C12N 2510/00; C12N 2513/00; C12N 2533/54; C12N 2740/10043

(57) ABSTRACT

[Problem] To directly convert somatic cells into cells that have an ability to form a stratified epithelial tissue capable of acting as the body outer skin and also have an ability to induce skin appendages.

[Solution] A production method of cells that have an ability to induce skin appendages, said method comprising a step for introducing into somatic cells at least one gene that is relatively strongly expressed in cells having the ability to induce skin appendages.

### FIELD OF THE INVENTION

The present invention relates to skin appendage-inducible cells induced from skin appendage-uninducible somatic cells, and a production method thereof. The present invention also relates to a cell preparation for regenerating skins and skin appendages, a method for treating skin ulcer, a method for treating alopecia, xeroderma, and asteatosis, as well as a platform for developing a skin and skin appendage regeneration method, which use the skin appendage-inducible cells. The present invention further relates to a cell preparation composition for inducing skin appendage-inducible cells from skin appendage-uninducible somatic cells.

### BACKGROUND ART

Skin appendages include hair follicles, sebaceous glands, sweat glands, and the like and play rolls such as protection from mechanical damage, heat retention, moisture retention, and thermoregulation. Skin appendages are formed by an interaction between epithelial tissues and mesenchymal tissues in the fetal period and during organogenesis (see Non-Patent Document 1).

Examples of representative pathological conditions caused by deletion, disorder, and dysfunction of skin appendages include alopecia and asteatosis. They cause not only thermoregulation disorder and itchiness due to dry skin but also aesthetic problems.

It is clinically difficult to regenerate lost skin appendages, and it is usually necessary to graft an existing appendage by skin grafting from another part of a body, flap surgery, or the like.

Thus, as a method of regeneration and neogenesis of skin appendages in an adult body, a method for reconstructing skin appendages by transplanting cells derived from fetal or neonatal animal skin (see Non-Patent Documents 2 and 3), a method for acquiring appendage-like structures by formation of organoids from pluripotent stem cells (see Non-Patent Documents 4 and 5), and the like have been developed. However, it has been difficult to achieve regeneration and neogenesis of a sufficient amount of skin appendages in a short period of time.

For neogenesis and regeneration of skin appendages, epithelial cells present on a surface layer of a body, mesenchymal cells present in a dermis or a subcutis, an interaction therebetween, and environmental factors play important roles (see Non-Patent Documents 6 and 7). Particularly, in recent years, studies using gene-recombinant animals have suggested that expression of genes related to dermal development, such as LEF1 gene and SHH gene, are particularly important as environmental factors for the regeneration of skin appendages, especially hair follicles (see Non-Patent Documents 8 to 10).

In the development process, once a cell lineage of a somatic cell has been established, the somatic cell continues to proliferate and differentiate within the cell lineage. In contrast, conversion of a cell lineage, which cannot occur under physiological conditions is referred to as direct conversion, and there have been reported technologies for direct conversion from skin fibroblasts to muscle cells (see Non-Patent Document 11), to nerve cells (see Non-Patent Document 12), to cardiomyocytes (see Non-Patent Document 13), to hepatocytes (see Non-Patent Document 14), to cells having an ability to form stratified squamous epithelium (see Patent Document 1 and Non-Patent Document 15), or the like.

With the background of aforementioned prior art, there is a strong desire to develop skin appendage-inducible cells that can be directly induced from skin appendage-uninducible somatic cells including skin fibroblasts and adipose-derived mesenchymal cells and can achieve regeneration and neogenesis of skin appendages, so as to provide a cell source for treatment of deletion, disorder, and dysfunction of skin appendages. However, there has been no report on direct conversion of skin appendage-inducible cells from skin appendage-uninducible somatic cells.

### PRIOR ART DOCUMENT

### Patent Document

Patent Document 1. WO2016/072105

### Non-Patent Document

Non-Patent Document 1. Lee J, Boscke R, Tang P C, Hartman B H, Heller S, Koehler K R. “Cell Research”, 2018, 22 (1), p 242-254
Non-Patent Document 2. Yuspa S H, Morgan D L, Walker R J, Bates R R. “J Invest Dermatol”, 1970, 55 (6), p 379-89.
Non-Patent Document 3. Worst P K, Mackenzie I C, Fusenig N E. “Cell Tissue Res”, 1982, 225 (1), p 65-77.
Non-Patent Document 4. Lee J, Boscke R, Tang P C, Hartman B H, Heller S, Koehler K R. “Cell Rep”, 2018, 22 (1), p 242-254.
Non-Patent Document 5. Lee J, Rabbani C C, Gao H, Steinhart M R, Woodruff B M, Pflum Z E, Kim A, Heller S, Liu Y, Shipchandler T Z, Koehler K R. “Nature”, 2020, 582 (7812), p 399-404.
Non-Patent Document 6. Sennett R, Wang Z, Rezza A, Grisanti L, Roitershtein N, Sicchio C, Mok K W, Heitman N J, Clavel C, Ma'ayan A, Rendl M. “Dev Cell”, 2015, 34 (5), p 577-591.
Non-Patent Document 7. Yang H, Adam R C, Ge Y, Hua Z L, Fuchs E. “Cell”, 2017, 169 (3), p 483-496.
Non-Patent Document 8. Lim C H, Sun Q, Ratti K, Lee S H, Zheng Y, Takeo M, Lee W, Rabbani P, Plikus M V, Cain J E, Wang D H, Watkins D N, Millar S, Taketo M M, Myung P, Cotsarelis G, Ito M. “Nat Commun”, 2018, 9 (1), 4903.
Non-Patent Document 9. Phan Q M, Fine G M, Salz L, Herrera G G, Wildman B, Driskell I M, Driskell R R. “Elife”, 2020, 9, e60066.
Non-Patent Document 10. Sun X, Are A, Annusver K, Sivan U, Jacob T, Dalessandri T, Joost S, Füllgrabe A, Gerling M, Kasper M. “Elife”, 2020, 9, e46756.
Non-Patent Document 11.Davis R L, Weintraub H, Lassar A B, “Cell”, 1987 Dec. 24, 51 (6), p 987-1000
Non-Patent Document 12. Vierbuchen T1, Ostermeier A, Pang Z P, Kokubu Y, Sudhof T C, Wernig M, “Nature”, 2010, 463 (7284), p 1035-1041
Non-Patent Document 13. Ieda M, Fu J D, Delgado-Olguin P, Vedantham V, Hayashi Y, Bruneau B G, Srivastava D, “Cell”, 2010, 142 (3), p 375-386
Non-Patent Document 14. Sekiya S, Suzuki A, “Nature”, 2011, 475 (7356), p 390-393
Non-Patent Document 15. Kurita M, Araoka T, Hishida T, O'Keefe D D, Takahashi Y, Sakamoto A, Sakurai M, Suzuki K, Wu J, Yamamoto M, Hernandez-Benitez R, Ocampo A, Reddy P, Shokhirev M N, Magistretti P, Núñez Delicado E, Eto H, Harii K, Izpisua Belmonte J C, “Nature”, 2018, 561 (7722), p 243-247

### SUMMARY OF THE INVENTION

### Problem to be Solved

To solve the aforementioned problems in the prior art, an object of the present invention is to directly convert skin appendage-uninducible somatic cells into skin appendage-inducible cells capable of inducing skin appendages such as hairs, hair follicles, and sebaceous glands. More specifically, an object of the present invention is to establish a technique for providing a cell source having an ability to form skin appendages such as hairs, hair follicles, and sebaceous glands. Also, an object of the present invention is to provide various applications of the skin appendage-inducible cells derived from these somatic cells.

### Solution to Problem

In order to solve the above problems, the present inventors adopted adipose tissue-derived mesenchymal cells as representative skin appendage-uninducible somatic cells and found that it was possible to produce a skin appendage-inducible cell population that can achieve regeneration and neogenesis of hairs, hair follicles, and sebaceous glands by transplanting cells in combination into a skin chamber attached to a back of an immunodeficient animal, through transfection of genes that are highly likely to characterize fetal skin epithelial cells and fetal skin mesenchymal cells having a vigorous skin appendage-reconstructing ability as well as mesenchymal cells constituting hair follicles.

The present invention provides the following aspects.
1. A method for producing a skin appendage-inducible cell, including a step of transfecting a transgene containing at least one gene that is relatively strongly expressed in a skin appendage-inducible cell into a skin appendage-uninducible somatic cell.
2. A method for producing a skin appendage-inducible cell, including a step of transfecting a transgene containing:
   (1) LEF1 gene; and
   (2) one, two, or three genes selected from DNP63A gene, GRHL2 gene, and TFAP2A gene into a skin appendage-uninducible somatic cell.
3. A method for producing a skin appendage-inducible cell, including a step of transfecting a transgene containing four genes: LEF1 gene, DNP63A gene, GRHL2 gene, and TFAP2A gene, into a skin appendage-uninducible somatic cell.
4. A method for producing a skin appendage-inducible cell, including a step of transfecting a transgene containing:
   (1) LEF1 gene; and
   (2) one, two, three, or four genes selected from DNP63A gene, GRHL2 gene, TFAP2A gene, and at least one MYC family genes into a skin appendage-uninducible somatic cell.
5. A method for producing a skin appendage-inducible cell, including a step of transfecting a transgene containing at least 5 genes: LEF1 gene, DNP63A gene, GRHL2 gene, TFAP2A gene, and at least one MYC family genes, into a skin appendage-uninducible somatic cell.
6. The method for producing the skin appendage-inducible cell according to the aspect 4 or 5, in which the transgene includes cMYC gene as the MYC family gene.
7. A method for producing a skin appendage-inducible cell, including a step of transfecting a transgene containing one or more genes selected from SHH gene, ETV1 gene, FOXD1 gene, PRDM1 gene, and LEF1 gene, into a skin appendage-uninducible somatic cell.
8. A method for producing a skin appendage-inducible cell, including a step of transfecting a transgene containing SHH gene into a skin appendage-uninducible somatic cell.
9. A method for producing a skin appendage-inducible cell, including a step of transfecting a transgene containing SHH gene and LEF1 gene into a skin appendage-uninducible somatic cell.
10. A method for producing a skin appendage-inducible cell, including a step of transfecting a transgene containing one, two, or three genes selected from ETV1 gene, PRDM1 gene, and FOXD1 gene into a skin appendage-uninducible somatic cell.
11. A method for producing a skin appendage-inducible cell, including a step of transfecting a transgene containing:
   (1) SHH gene; and
   (2) one or more genes selected from ETV1 gene, PRDM1 gene, FOXD1 gene, and LEF1 gene into a skin appendage-uninducible somatic cell.
12. A method for producing a skin appendage-inducible cell, including a step of transfecting a transgene containing:
   (1) ETV1 gene and PRDM1 gene;
   (2) FOXD1 gene and PRDM1 gene; or
   (3) ETV1 gene, PRDM1 gene, and FOXD1 gene
      into a skin appendage-uninducible somatic cell.
13. The method for producing the skin appendage-inducible cell according to any one of the aspects 7 to 12, in which a plurality of cells having different transgenes are prepared for transfecting multiple types of the genes.
14. The method for producing the skin appendage-inducible cell according to any one of the aspects 1 to 13, in which the somatic cell is derived from human.
15. The method for producing the skin appendage-inducible cell according to any one of the aspects 1 to 14, in which the somatic cell is a skin fibroblast, a subcutaneous adipose tissue-derived stromal cell (subcutaneous adipocyte), an embryonic fibroblast, an adipocyte, a muscle cell, an osteoblast, a cartilage cell, a monocyte in circulating blood, or somatic cells differentiated from an ES cell, a mesenchymal stem cell, or the like.
16. A skin appendage-inducible cell produced by the production method according to any one of the aspects 1 to 15.
17. A cell preparation containing the skin appendage-inducible cell according to the aspect 16.
18. A cell preparation containing:
   (1) a first skin appendage-inducible cell produced by a step of transfecting a transgene containing SHH gene or containing SHH gene and LEF1 gene into a skin appendage-uninducible somatic cell; and
   (2) a second skin appendage-inducible cell produced by a step of transfecting a transgene containing one or more genes selected from ETV1 gene, PRDM1 gene, and FOXD1 gene into a skin appendage-uninducible somatic cell.
19. The cell preparation according to the aspect 18, in which the transgene of the second cell contains:
   (1) ETV1 gene and PRDM1 gene;
   (2) FOXD1 gene and PRDM1 gene; or
   (3) ETV1 gene, PRDM1 gene, and FOXD1 gene.
20. The cell preparation according to any one of the aspects 17 to 19, containing a scaffold material.
21. The cell preparation according to the aspect 20, in which the scaffold material is collagen.
22. The cell preparation according to any one of the aspects 17 to 21, in which the cell preparation is for a purpose of regenerating skin tissues or hairs.
23. The cell preparation according to any one of the aspects 17 to 22, in which the cell preparation is a sheet-like structure or a cell aggregate having a three-dimensional structure.
24. A non-human mammal having a formed skin appendage, the non-human mammal being produced by administering the skin appendage-inducible cell according to the aspect 16 or the cell preparation according to any one of the aspects 17 to 23 into a non-human mammal to form the skin appendage from the skin appendage-inducible cell in a body of the mammal.
25. A method for determining a drug efficacy of a test substance, including a step of administering the test substance to the non-human mammal according to the aspect 24 to determine the drug efficacy of the test substance on skin and/or a skin appendage tissue.
26. A method for determining an influence of an external factor, including a step of loading the non-human mammal according to the aspect 24 with a stress such as an anticancer drug and a radiation to determine a stress on skin and/or a skin appendage tissue.
27. A skin appendage-inducible cell preparation composition containing a transgene used in the production method according to any one of the aspects 1 to 12.
28. The cell preparation composition according to the aspect 27, containing the transgene in a form transfectable into a somatic cell.
29. A skin tissue and skin appendage-like tissue produced by culturing the skin appendage-inducible cell according to the aspect 16.
30. A method for analyzing a drug efficacy of a test substance, including a step of administering the test substance to the skin tissue and/or skin appendage-like tissue according to the aspect 29 to analyze the drug efficacy of the test substance on the skin tissue and/or skin appendage-like tissue.
31. A method for analyzing an influence of an external factor, including a step of loading the skin tissue and/or skin appendage-like tissue according to the aspect 29 with a stress such as an anticancer drug or a radiation to analyze a stress on the skin tissue and/or skin appendage-like tissue.
32. A kit of a vector specialized for transfecting the gene according to any one of the aspects 1 to 12 into a somatic cell.
33. A method for evaluating a skin appendage-inducing ability of a cell, using the skin appendage-inducible cell according to the aspect 16.
34. A vector for a purpose of producing a skin appendage-inducible cell, in which the vector expresses LEF1 gene, DNP63A gene, GRHL2 gene, TFAP2A gene, and cMYC gene.
35. A vector for a purpose of producing a skin appendage-inducible cell, in which the vector expresses SHH gene, ETV1 gene, PRDM1 gene, FOXD1 gene, and LEF1 gene.
36. A kit of a vector including:
   a first vector for a purpose of producing a skin appendage-inducible cell, the first vector expressing SHH gene, or SHH gene and LEF1 gene; and
   a second vector for a purpose of producing a skin appendage-inducible cell, the second vector expressing one or more genes selected from ETV1 gene, PRDM1 gene, and FOXD1 gene.
37. The kit of the vector according to the aspect 36, in which the second vector expresses:
   (1) ETV1 gene and PRDM1 gene;
   (2) FOXD1 gene and PRDM1 gene; or
   (3) ETV1 gene, PRDM1 gene, and FOXD1 gene.
38. The vector according to the aspect 34 or 35, or the kit of the vector according to the aspect 32, 36, or 37, in which the vector is a viral vector.
39. A cell preparation containing a skin appendage-inducible cell produced by the production method according to any one of the aspects 2 to 6 and a skin appendage-inducible mesenchymal cell.
40. The cell preparation according to the aspect 39, in which the skin appendage-inducible mesenchymal cell is a skin appendage-inducible cell produced by the production method according to any one of the aspects 7 to 12.
41. A cell preparation containing a skin appendage-inducible cell produced by the production method according to any one of the aspects 7 to 12 and a skin appendage-inducible epithelial cell.
42. The cell preparation according to the aspect 41, in which the skin appendage-inducible epithelial cell is a skin appendage-inducible cell produced by the production method according to any one of the aspects 2 to 6.

### Advantageous Effects of Invention

The present invention makes it possible to provide cells capable of forming a tissue having characteristics equivalent to that of skin including skin appendages. Such cells that reconstruct the skin appendages can provide a medical means or a cell preparation effective for treating pathological conditions caused by deletion and dysfunction of skin appendages such as alopecia, xeroderma, and asteatosis, as well as skin ulcers such as burns, trauma, decubitus, diabetic ulcer, and skin ulcer due to peripheral circulatory failure. The present invention can contribute to pathological elucidation or treatment of diseases by preparing a skin appendage-inducible cell from a patient's somatic cell (it is not limited to skin-derived cells but can be exemplified by blood and adipose tissue stromal cells) to variously analyze the prepared cell. In particular, the skin appendage-inducible cells prepared from human somatic cells are suitable as a material for drug discovery and drug development also from the standpoint of confirming drug efficacy. Use of the skin appendage-inducible cell having contradictory characteristics of self-replicability and differentiation can contribute to elucidation of pathological conditions originating from stem cells, such as cancer and sarcoma. In patients in a pathological condition lacking in skin appendages such as a pathological condition of skin and skin appendage defect like a skin ulcer surface, a condition of hypoactive skin appendage and functions thereof due to age-related changes, and a condition after healing from skin ulcer, the skin appendage-inducible cells can be used as a treatment means and a therapeutic method for inducing regeneration and neogenesis of skin appendages to satisfy the skin appendage function, by transplantation of the cells prepared in the present invention, or application of the present invention to somatic cells of a living body.

### BRIEF DESCRIPTION OF THE DRAWINGS

FIG. 1 is a scheme illustrating a transplantation test on adult mouse skin-derived cells, showing that epithelial and mesenchymal cells are isolated and cultured from adult mice and transplanted into immunodeficient animals.

FIG. 2 is a photograph presenting a result of the transplantation test on the adult mouse skin-derived cells (Reference Example 1), showing that skin appendages were not regenerated by the transplantation of the adult mouse skin-derived epithelial and mesenchymal cells.

FIG. 3 is a scheme illustrating a transplantation test on neonatal mouse skin-derived cells, showing that epithelial and mesenchymal cells are isolated and cultured from skin of neonatal mice and transplanted into immunodeficient animals.

FIG. 4 is a photograph presenting a result of the transplantation test on the neonatal mouse skin-derived cells (Reference Example 2), showing that skin appendages were regenerated by the transplantation of the neonatal mouse skin-derived epithelial and mesenchymal cells.

FIG. 5 is a scheme illustrating a transplantation test on induced epithelial cells prepared by transfection into adult mouse adipose tissue cell-derived mesenchymal cells, and neonatal mouse skin-derived mesenchymal cells.

FIG. 6 is a photograph presenting a result of a transplantation test (Comparative Example 1) on induced epithelial cells prepared by transfecting DNP63A, GRHL2, TFAP2A, and cMYC into adult mouse adipose tissue cell-derived mesenchymal cells, and neonatal mouse skin-derived mesenchymal cells, showing that skin appendages were not regenerated by the transplantation of the induced epithelial cells prepared by transfecting DNP63A, GRHL2, TFAP2A, and cMYC into adult mouse adipose tissue cell-derived mesenchymal cells, and the neonatal mouse skin-derived mesenchymal cells.

FIG. 7 is a photograph presenting a result of a test in which DNP63A, GRHL2, TFAP2A, c-MYC, and SHH are transfected into adult mouse adipose-derived mesenchymal cells (Comparative Example 2), showing that epithelial cells could not be induced by transfecting DNP63A, GRHL2, TFAP2A, c-MYC, and SHH into the adult mouse adipose-derived mesenchymal cells.

FIG. 8 is a photograph presenting a result of a test in which DNP63A, GRHL2, TFAP2A, c-MYC, and FOXD1 are transfected into adult mouse adipose-derived mesenchymal cells (Comparative Example 3), showing that epithelial cells can be induced by transfecting DNP63A, GRHL2, TFAP2A, c-MYC, and FOXD1 into the adult mouse adipose-derived mesenchymal cells.

FIG. 9 is a photograph presenting a result of a transplantation test (Comparative Example 3) on induced epithelial cells prepared by transfecting DNP63A, GRHL2, TFAP2A, cMYC, and FOXD1 into adult mouse adipose tissue cell-derived mesenchymal cells, and neonatal mouse skin-derived mesenchymal cells, showing that skin appendages were not regenerated by the transplantation of the induced epithelial cells prepared by transfecting DNP63A, GRHL2, TFAP2A, cMYC, and FOXD1 into adult mouse adipose tissue cell-derived mesenchymal cells, and the neonatal mouse skin-derived mesenchymal cells.

FIG. 10 is a photograph presenting a result of a test in which DNP63A, GRHL2, TFAP2A, c-MYC, and LEF1 are transfected into adult mouse adipose-derived mesenchymal cells (Example 1), showing that epithelial cells can be induced by transfecting DNP63A, GRHL2, TFAP2A, c-MYC, and LEF1 into the adult mouse adipose-derived mesenchymal cells.

FIG. 11 is a photograph presenting a result of a transplantation test (Example 1) on induced epithelial cells prepared by transfecting DNP63A, GRHL2, TFAP2A, cMYC, and LEF1 into adult mouse adipose tissue cell-derived mesenchymal cells, and neonatal mouse skin-derived mesenchymal cells, showing that skin appendages can be regenerated by the transplantation of the induced epithelial cells prepared by transfecting DNP63A, GRHL2, TFAP2A, cMYC, and LEF1 into adult mouse adipose tissue cell-derived mesenchymal cells, and the neonatal mouse skin-derived mesenchymal cells.

FIG. 12 is a graph presenting changes in expression of marker genes (PROM1 gene, CRABP1 gene, and VCAN gene) in transfection of a candidate factor for inducing skin appendage-inducible mesenchymal cells into cultured human fibroblasts. The “ALL” represents changes in expression of the marker genes in simultaneous transfection of all candidate factors. Each bar in the graphs represents a mean value, and each error bar represents a standard deviation. Candidate factors showing statistically significant changes compared to NC (control without transfection) are indicated by * (*p<0.05, **p<0.01, ***p<0.001).

FIG. 13 is a graph presenting numbers of positive cells in alkaline phosphatase staining after transfection of a candidate factor for inducing skin appendage-inducible mesenchymal cells into cultured human fibroblasts. The “ALL” represents the number of positive cells in simultaneous transfection of all candidate factors. Each bar in the graphs represents a mean value, and each error bar represents a standard deviation. Candidate factors showing statistically significant changes compared to NC (control without transfection) are indicated by * (*p<0.05, **p<0.01, ***p<0.001).

FIG. 14 is a graph presenting numbers of alkaline phosphatase-positive cells in combined transfection of a plurality of candidate factors for inducing skin appendage-inducible mesenchymal cells into cultured human fibroblasts, which candidate factors have been determined to be highly effective on the basis of on the marker gene expression and alkaline phosphatase expression results obtained from transfection of each of the candidate factors. The “+” represents that the factors have been added, and the “−” represents that the factors have not been added. Each bar in the graphs represents a mean value, and each error bar represents a standard deviation. Candidate factors showing statistically significant changes compared to NC (control without transfection: the rightmost result) are indicated by * (*p<0.05, **p<0.01, ***p<0.001).

FIG. 15 is a scheme illustrating a transplantation test on induced epithelial cells prepared by transfecting DNP63A, GRHL2, TFAP2A, c-MYC, and LEF1 into adult mouse adipose-derived mesenchymal cells, and induced mesenchymal cells prepared by transfecting candidate factors for inducing skin appendage-inducible mesenchymal cells into adult mouse adipose-derived mesenchymal cells.

FIG. 16 is a photograph presenting a result of a transplantation test (Example 2-1) on induced epithelial cells prepared by transfecting DNP63A, GRHL2, TFAP2A, c-MYC, and LEF1 into adult mouse adipose-derived mesenchymal cells, induced mesenchymal cells prepared by transfecting ETV1, FOXD1, and PRDM1 into adult mouse adipose-derived mesenchymal cells, and induced mesenchymal cells prepared by transfecting SHH and LEF1 into adult mouse adipose-derived mesenchymal cells, showing that skin appendages can be regenerated by the transplantation of these induced cells.

FIG. 17 is a cross-sectional photograph presenting a result of a transplantation test (Example 2-1) on induced epithelial cells prepared by transfecting DNP63A, GRHL2, TFAP2A, c-MYC, and LEF1 into adult mouse adipose-derived mesenchymal cells, induced mesenchymal cells prepared by transfecting ETV1, FOXD1, and PRDM1 into adult mouse adipose-derived mesenchymal cells, and induced mesenchymal cells prepared by transfecting SHH and LEF1 into adult mouse adipose-derived mesenchymal cells. In this figure, mature hair shafts (arrows 1), hair matrix-like tissues (arrows 2), and sebaceous gland-like tissues (arrow 3) can be observed.

FIG. 18 is a photograph presenting a result of a transplantation test (Example 2-2) on induced epithelial cells prepared by transfecting DNP63A, GRHL2, TFAP2A, c-MYC, and LEF1 into adult mouse adipose-derived mesenchymal cells, induced mesenchymal cells prepared by transfecting ETV1, FOXD1, and PRDM1 into adult mouse adipose-derived mesenchymal cells, and induced mesenchymal cells prepared by transfecting SHH into adult mouse adipose-derived mesenchymal cells, showing that skin appendages can be regenerated by the transplantation of these induced cells.

FIG. 19 is a photograph presenting a result of a transplantation test (Example 2-3) on induced epithelial cells prepared by transfecting DNP63A, GRHL2, TFAP2A, c-MYC, and LEF1 into adult mouse adipose-derived mesenchymal cells, induced mesenchymal cells prepared by transfecting ETV1 and FOXD1 into adult mouse adipose-derived mesenchymal cells, and induced mesenchymal cells prepared by transfecting SHH and LEF1 into adult mouse adipose-derived mesenchymal cells, showing that skin appendages can be regenerated by the transplantation of these induced cells.

FIG. 20 is a photograph presenting a result of a transplantation test (Example 2-4) on induced epithelial cells prepared by transfecting DNP63A, GRHL2, TFAP2A, c-MYC, and LEF1 into adult mouse adipose-derived mesenchymal cells, induced mesenchymal cells prepared by transfecting FOXD1 and PRDM1 into adult mouse adipose-derived mesenchymal cells, and induced mesenchymal cells prepared by transfecting SHH and LEF1 into adult mouse adipose-derived mesenchymal cells, showing that skin appendages can be regenerated by the transplantation of these induced cells.

FIG. 21 is a photograph presenting a result of a transplantation test (Example 2-5) on induced epithelial cells prepared by transfecting DNP63A, GRHL2, TFAP2A, c-MYC, and LEF1 into adult mouse adipose-derived mesenchymal cells, induced mesenchymal cells prepared by transfecting ETV1 and PRDM1 into adult mouse adipose-derived mesenchymal cells, and induced mesenchymal cells prepared by transfecting SHH and LEF1 into adult mouse adipose-derived mesenchymal cells, showing that skin appendages can be regenerated by the transplantation of these induced cells.

FIG. 22 is a photograph presenting a result of a transplantation test (Example 2-6) on induced epithelial cells prepared by transfecting DNP63A, GRHL2, TFAP2A, c-MYC, and LEF1 into adult mouse adipose-derived mesenchymal cells, and induced mesenchymal cells prepared by transfecting ETV1, FOXD1, PRDM1, and LEF1 into adult mouse adipose-derived mesenchymal cells, showing that skin appendages can be regenerated by the transplantation of these induced cells.

FIG. 23 is a photograph presenting a result of a transplantation test (Example 2-7) on induced epithelial cells prepared by transfecting DNP63A, GRHL2, TFAP2A, c-MYC, and LEF1 into adult mouse adipose-derived mesenchymal cells, and induced mesenchymal cells prepared by transfecting ETV1, FOXD1, PRDM1, SHH, and LEF1 into adult mouse adipose-derived mesenchymal cells, showing that skin appendages can be regenerated by the transplantation of these induced cells.

FIG. 24 is a photograph presenting a result of a transplantation test (Example 2-8) on induced epithelial cells prepared by transfecting DNP63A, GRHL2, TFAP2A, c-MYC, and LEF1 into adult mouse adipose-derived mesenchymal cells, and induced mesenchymal cells prepared by transfecting SHH and LEF1 into adult mouse adipose-derived mesenchymal cells. This figure shows that skin appendages can be regenerated by the transplantation of these induced cells.

FIG. 25 is a photograph presenting a result of a transplantation test (Example 2-9) on induced epithelial cells prepared by transfecting DNP63A, GRHL2, TFAP2A, c-MYC, and LEF1 into adult mouse adipose-derived mesenchymal cells, induced mesenchymal cells prepared by transfecting PRDM1 into adult mouse adipose-derived mesenchymal cells, and induced mesenchymal cells prepared by transfecting SHH and LEF1 into adult mouse adipose-derived mesenchymal cells, showing that skin appendages can be regenerated by the transplantation of these induced cells.

FIG. 26 is a photograph presenting a result of a transplantation test (Example 2-10) on induced epithelial cells prepared by transfecting DNP63A, GRHL2, TFAP2A, c-MYC, and LEF1 into adult mouse adipose-derived mesenchymal cells, induced mesenchymal cells prepared by transfecting FOXD1 and PRDM1 into adult mouse adipose-derived mesenchymal cells, and induced mesenchymal cells prepared by transfecting SHH into adult mouse adipose-derived mesenchymal cells, showing that skin appendages can be regenerated by the transplantation of these induced cells. (A) is an overall image of the transplanted area, and (B) is an enlarged image of the hair growth area.

FIG. 27 is a photograph presenting a result of a transplantation test (Example 2-11) on induced epithelial cells prepared by transfecting DNP63A, GRHL2, TFAP2A, c-MYC, and LEF1 into adult mouse adipose-derived mesenchymal cells, induced mesenchymal cells prepared by transfecting PRDM1 into adult mouse adipose-derived mesenchymal cells, and induced mesenchymal cells prepared by transfecting SHH into adult mouse adipose-derived mesenchymal cells, showing that skin appendages can be regenerated by the transplantation of these induced cells. (A) is an overall image of the transplanted area, and (B) is an enlarged image of the hair growth area.

FIG. 28 is a photograph presenting a result of a transplantation test (Example 2-12) on induced epithelial cells prepared by transfecting DNP63A, GRHL2, TFAP2A, c-MYC, and LEF1 into adult mouse adipose-derived mesenchymal cells, induced mesenchymal cells prepared by transfecting FOXD1 into adult mouse adipose-derived mesenchymal cells, and induced mesenchymal cells prepared by transfecting SHH into adult mouse adipose-derived mesenchymal cells, showing that skin appendages can be regenerated by the transplantation of these induced cells. (A) is an overall image of the transplanted area, and (B) is an enlarged image of the hair growth area.

FIG. 29 is a photograph presenting a result of a transplantation test (Example 2-13) on induced epithelial cells prepared by transfecting DNP63A, GRHL2, TFAP2A, c-MYC, and LEF1 into adult mouse adipose-derived mesenchymal cells, and induced mesenchymal cells prepared by transfecting SHH into adult mouse adipose-derived mesenchymal cells, showing that skin appendages can be regenerated by the transplantation of these induced cells. (A) is an overall image of the transplanted area, and (B) is an enlarged image of the hair growth area.

FIG. 30 is a photograph presenting a result of a transplantation test (Example 2-14) on induced epithelial cells prepared by transfecting DNP63A, GRHL2, TFAP2A, c-MYC, and LEF1 into adult mouse adipose-derived mesenchymal cells, and induced mesenchymal cells prepared by transfecting ETV1, FOXD1, and PRDM1 into adult mouse adipose-derived mesenchymal cells, showing that hair follicle-like structures and mature hair shafts can be formed under the skin by the transplantation of these induced cells. (A) is a photograph of an appearance on day 28 (D28), in which the dotted line represents a site from which tissue images were taken. (B) is a cross-sectional photograph of a hematoxylin eosin-stained (HE) tissue, in which the dotted square indicates the location of (C). (C) is an enlarged image. (D) and (E) are tissue images of slices continuous to (C). Arrows indicate mature hair shafts, showing that a series of contiguous shafts were observed in the slices.

FIG. 31 is a photograph presenting a result of a transplantation test (Example 3) on induced epithelial cells prepared by transfecting DNP63A, GRHL2, TFAP2A, and LEF1 into adult mouse adipose-derived mesenchymal cells, induced mesenchymal cells prepared by transfecting ETV1, FOXD1, and PRDM1 into adult mouse adipose-derived mesenchymal cells, and induced mesenchymal cells prepared by transfecting SHH and LEF1 into adult mouse adipose-derived mesenchymal cells, showing that skin appendages can be regenerated by the transplantation of these induced cells. (A) is an overall image of the transplanted area, and (B) is an enlarged image of the hair growth area.

FIG. 32 is a graph presenting numbers of newly grown hairs observed on transplantation sites as a result of a process in which induced epithelial cells prepared by transfecting DNP63A, GRHL2, TFAP2A, c-MYC, and LEF1 into adult mouse adipose-derived mesenchymal cells, and one or two types of mesenchymal cells prepared by transfecting a combination of several genes that are relatively strongly expressed in skin appendage-inducible mesenchymal cells are transplanted in combination into a chamber attached to a back of an immunodeficient animal. The graph presents results of two experiments for each condition.

FIG. 33 is a photograph presenting (A) a mold of silicone chambers, (B) prepared silicone chambers, and (C) a mouse with a back to which a silicone chamber is fixed by suture.

### DETAILED DESCRIPTION OF THE INVENTION

### 1. Production Method and Use of Skin Appendage-Inducible Cell

In the present invention, the “skin appendage-inducible cells” refer to cells capable of serving as progenitor cells or stem cells of a stratified epithelium that protects an inside of a body from external factors such as mechanical disorder and infection on sites in contact with the external environment, such as epidermis (in other words, epithelial stem cells). The “skin appendage-inducible cells” mean cells that reconstruct skin appendages such as hair follicles and sebaceous glands when transplanted with appropriate mesenchymal cells, and/or mesenchymal cells that reconstruct skin appendages such as hair follicles and sebaceous glands when transplanted with appropriate epithelial cells, and further include cells derived from fetal or neonatal skin or cells derived from adult hair follicles and appendages. Furthermore, the skin appendage-inducible cells can also form a stratified squamous epithelium.

The method for producing skin appendage-inducible cells according to the present invention characteristically includes a step of transfecting at least one type of gene that is relatively strongly expressed in skin appendage-inducible cells (e.g., skin cells derived from a fetus and a neonate, or cells constituting skin appendages such as hair follicles and sebaceous glands), into skin appendage-uninducible somatic cells. Hereinafter, the gene to be transfected will be referred to as a “transgene”. As the transgene, a plural types of genes that are relatively strongly expressed in skin appendage-inducible cells may be used, or the genes that are relatively strongly expressed in skin appendage-inducible cells may be combined with genes that are not relatively strongly expressed in skin appendage-inducible cells.

Herein, the “transgene” includes not only protein-coding genes but also non-coding RNAs such as microRNAs. The “gene that is relatively strongly expressed in skin appendage-inducible cells” refers to a gene that has been confirmed to be more-highly expressed in skin appendage-inducible cells than in skin appendage-uninducible cells such as adult-derived keratinocytes, skin fibroblasts, and adipose-derived mesenchymal cells, by a method for quantitatively evaluate a gene expression level such as the real-time PCR method, the microarray method, and the RNA sequencing. The “gene that is not relatively strongly expressed” refers to a gene that is less expressed in skin appendage-inducible cells than in skin appendage-uninducible cells.

The gene that is relatively strongly expressed in skin appendage-inducible epithelial cells includes at least the following protein-coding genes: DNP63A gene, GRHL2 gene, TFAP2A gene, cMYC gene, and LEF1 gene.

The gene that is relatively strongly expressed in skin appendage-inducible mesenchymal cells includes at least the following protein-coding genes: SOX2 gene, LEF1 gene, HOXC4 gene, HOXC9 gene, HOXC13 gene, JARID2 gene, HEY1 gene, HEY2 gene, FOXO1 gene, FOXD1 gene, EGR3 gene, MEF2C gene, LHX2 gene, PRRX1 gene, PRRX2 gene, CREB3 gene, ETV1 gene, TFAP2A gene, cMYC gene, TBX6 gene, MSX2 gene, SHH gene, and PRDM1 gene.

All genes used in the present invention have known base sequences (Table 1). In the present specification, NCBI is an abbreviation of U.S. National Center for Biotechnology Information, and the accession Nos. in Table 1 are numbers registered in the database provided by NCBI.

TABLE 1

| | Human | Mouse | | Human | Mouse |
|---|---|---|---|---|---|
| | Reference | Reference | | Reference | Reference |
| Gene | sequences | sequences | Gene | sequences | sequences |
| DNP63A | NM_001114980.2 | NM_011641.2 | MEF2C | NM_001131005.2 | NM_001170537.1 |
| GRHL2 | NM_001330593.2 | NM_026496.4 | | NM_001193347.1 | NM_001347564.1 |
| | NM_024915.4 | | | NM_001193348.1 | NM_001347566.1 |
| TFAP2A | NM_001032280.3 | NM_001122948.2 | | NM_001193349.3 | NM_001347567.1 |
| | NM_001042425.3 | NM_001301674.2 | | NM_001193350.2 | NM_001347568.1 |
| | NM_001372066.1 | NM_011547.5 | | NM_001308002.3 | NM_001347569.1 |
| cMYC | NM_001354870.1 | NM_001177352.1 | | NM_001363581.2 | NM_001347571.1 |
| | NM_002467.6 | NM_001177353.1 | | NM_001364329.2 | NM_001347572.1 |
| | | NM_001177354.1 | | NM_001364330.2 | NM_001347573.1 |
| | | NM_010849.4 | | NM_001364331.2 | NM_001347574.1 |
| LEF1 | NM_001130713.3 | NM_001276402.1 | | NM_001364332.2 | NM_001347575.1 |
| | NM_001130714.3 | NM_001276403.1 | | NM_001364333.2 | NM_001347576.1 |
| | NM_001166119.2 | NM_001379059.1 | | NM_001364334.2 | NM_001347577.1 |
| | NM_016269.5 | NM_001379060.1 | | NM_001364335.2 | NM_001347578.1 |
| | | NM_001379061.1 | | NM_001364336.2 | NM_001347579.1 |
| | | NM_001379062.1 | | NM_001364337.2 | NM_001347580.1 |
| | | NM_010703.4 | | NM_001364338.2 | NM_001347581.1 |
| SOX2 | NM_003106.4 | NM_011443.4 | | NM_001364339.2 | NM_025282.3 |
| HOXA9 | NM_152739.4 | NM_001277238.1 | | NM_001364340.2 | |
| | | NM_010456.3 | | NM_001364341.2 | |
| HOXC4 | NM_014620.6 | NM_013553.2 | | NM_001364342.2 | |
| | NM_153633.3 | | | NM_001364343.2 | |
| HOXC9 | NM_006897.3 | NM_008272.3 | | NM_001364344.2 | |
| HOXC13 | NM_017410.3 | NM_010464.2 | | NM_001364345.2 | |
| JARID2 | NM_001267040.1 | NM_001205043.1 | | NM_001364346.2 | |
| | NM_004973.4 | NM_001205044.1 | | NM_001364347.2 | |
| | | NM_001360281.1 | | NM_001364348.2 | |
| | | NM_021878.3 | | NM_001364349.2 | |
| HEY1 | NM_001040708.2 | NM_010423.2 | | NM_001364350.2 | |
| | NM_001282851.2 | | | NM_001364352.2 | |
| | NM_012258.4 | | | NM_001364353.2 | |
| HEY2 | NM_012259.3 | NM_013904.1 | | NM_001364354.2 | |
| FOXO1 | NM_002015.4 | NM_019739.3 | | NM_001364355.2 | |
| FOXD1 | NM_004472.3 | NM_008242.2 | | NM_001364356.2 | |
| EGR3 | NM_001199880.2 | NM_001289925.2 | | NM_001364357.2 | |
| | NM_001199881.2 | NM_001289927.1 | | NM_002397.5 | |
| | NM_004430.3 | NM_018781.4 | CREB3 | NM_006368.5 | NM_013497.4 |

| | Human | Mouse |
|---|---|---|
| | Reference | Reference |
| Gene | sequences | sequences |
| LHX2 | NM_004789.4 | NM_001290646.1 |
| | | NM_010710.4 |
| PRRX1 | NM_006902.5 | NM_001025570.1 |
| | NM_022716.4 | NM_011127.2 |
| | | NM_175686.3 |
| ETV1 | NM_001163147.2 | NM_001163154.1 |
| | NM_001163148.2 | NM_001347379.1 |
| | NM_001163149.2 | NM_007960.5 |
| | NM_001163150.2 | |
| | NM_001163151.2 | |
| | NM_001163152.2 | |
| | NM_001370555.1 | |
| | NM_001370556.1 | |
| | NM_004956.5 | |
| cMYC | NM_001354870.1 | NM_001177352.1 |
| | NM_002467.6 | NM_001177353.1 |
| | | NM_001177354.1 |
| | | NM_010849.4 |
| TBX6 | NM_004608.4 | NM_011538.2 |
| MSX2 | NM_001363626.2 | NM_013601.2 |
| | NM_002449.5 | |
| SHH | NM_000193.4 | NM_009170.3 |
| | NM_001310462.2 | |
| PRDM1 | NM_001198.4 | NM_007548.4 |
| | NM_182907.3 | |

Many of the origins of these genes are commonly found in mammals including human, and genes derived from any mammalian can be used, but it is desirable to select a gene appropriately depending on an origin of a somatic cell into which the gene is transfected. For example, if human-derived somatic cells are used, it is desirable that the transgene is derived from human. The transgene may be, besides wild-type genes, a mutant gene in which several (e.g., 1 to 10, preferably 1 to 6, more preferably 1 to 4, even more preferably 1 to 3, and particularly preferably 1 or 2) amino acids in amino acid sequences of a gene product are substituted, deleted, and/or inserted, and which encodes a mutant gene product having a function equivalent to that of the wild-type gene product. It is possible to use a sequence of which codons are modified and optimized so as to encode the same amino acid as the amino acid encoded by each gene.

In the present invention, the transgene can be prepared according to a routine method on the basis of known sequence information. For example, a cDNA of a target gene can be prepared by extracting an RNA from a mammalian-derived cell and cloning the RNA according to a routine method. The transgene can be synthesized as an artificial gene. When synthesizing an artificial gene, codons can be optimized according to an original animal of a somatic cell to be transfected.

In the present invention, the type of the “somatic cell” to be induced into the skin appendage-inducible cell is not particularly limited, and a somatic cell derived from any tissue or region can be used. Examples of somatic cells used in the present invention include cells derived from tissues such as skin, subcutaneous adipose, muscle, placenta, bone, cartilage, blood, and corneal stroma, more specifically, skin fibroblasts, subcutaneous adipose tissue-derived stromal cells (subcutaneous adipocytes), embryonic fibroblasts, adipocytes, muscle cells, osteoblasts, cartilage cells, and monocytes in circulating blood. Above all, skin-derived cells, subcutaneous adipose-derived cells, or blood-derived cells are preferable, and skin fibroblasts, subcutaneous adipose tissue-derived stromal cells, and monocytes in circulating blood are preferable, from the viewpoint of decreasing invasiveness to a living body and achieving more efficient production of the skin appendage-inducible cells. Also, in terms of reduction of patient's burden and stable cell availability, it is clinically advantageous to enable selection of a material from various cells, particularly use of cells that are easily available such as skin-derived cells, subcutaneous adipose-derived cells, and monocytes in circulating blood. A commercially available product, and somatic cells differentiated from ES cells, mesenchymal stem cells, or the like can also be used as the somatic cells.

The somatic cells are selected as appropriate from those derived from mammals such as human, mouse, rat, hamster, rabbit, cat, dog, sheep, pig, cow, goat, and monkey depending on an intended use for the skin appendage-inducible cells. However, human-derived somatic cells are suitable for use in treatment, pathological elucidation, drug efficacy evaluation, or the like, on human subjects. When human-derived somatic cells are used, the cells may be derived from any of a fetus, an infant, a child, and an adult. When the skin appendage-inducible cells are used for the purpose of treatment, pathological elucidation, or drug efficacy evaluation, or the like on a human subject, it is desirable to use somatic cells collected from the patient.

The transgene can be transfected into somatic cells by a method ordinarily used for transfection of animal cells. Specific examples of the method for transfecting the transgene into the somatic cells include: a method using a vector; a calcium phosphate method; a lipofection method; an electroporation method; and a microinjection method. Above all, the method using a vector is preferable from the viewpoint of the transfection efficiency. When the transgene is transfected into the somatic cells using a vector, a viral vector, a non-viral vector (including a plasmid (DNA) vector and an mRNA vector), an artificial virus, or the like can be used. Above all, viral vectors such as an adeno-associated virus, a retrovirus, and lentivirus are suitably used from the viewpoint of safety. Note that, when a vector is used and there are a plurality of transgenes, each transgene may be incorporated into separate vectors, or two or more types of transgenes may be incorporated into one vector.

The somatic cells into which the transgene has been transfected as described above can be induced into the skin appendage-inducible cells.

It is also possible to induce the skin appendage-inducible cells from somatic cells in a living body by transfecting the transgene into the in vivo somatic cells using the aforementioned gene transfection means or vector. The transgene may be a gene that is relatively strongly expressed in skin appendage-inducible epithelial cells or a gene that is relatively strongly expressed in skin appendage-inducible mesenchymal cells, but it is preferable to transfect both of these genes.

### [Induced Epithelial Cell]

The method for producing skin appendage-inducible cells, particularly epithelial cells according to the present invention preferably includes a step of transfecting a transgene including: (1) LEF1 gene; and (2) one, two, or three genes selected from DNP63A gene, GRHL2 gene, and TFAP2A gene into skin appendage-uninducible somatic cells. In particular, the method for producing skin appendage-inducible epithelial cells according to the present invention preferably includes a step of transfecting a transgene including four genes: LEF1 gene, DNP63A gene, GRHL2 gene, and TFAP2A gene, into skin appendage-uninducible somatic cells.

Furthermore, the method for producing skin appendage-inducible cells, particularly epithelial cells according to the present invention preferably includes a step of transfecting a transgene including: (1) LEF1 gene; and (2) one, two, three, or four genes selected from DNP63A gene, GRHL2 gene, TFAP2A gene, and at least one MYC family genes into skin appendage-uninducible somatic cells. In particular, the method for producing skin appendage-inducible epithelial cells according to the present invention preferably includes a step of transfecting a transgene including at least 5 genes: LEF1 gene, DNP63A gene, GRHL2 gene, TFAP2A gene, and at least one MYC family genes, into skin appendage-uninducible somatic cells.

The Myc family genes include c-Myc, N-Myc, L-Myc, and the like. Each of these Myc family genes may be used alone or in combination with a plurality of the genes. It is preferable to use c-Myc gene as the Myc family gene.

As confirmed in the method for inducing the stratified squamous epithelium-forming ability in Non-Patent Document 15, the skin appendage-inducible epithelial cells can also be produced by further adding other genes in addition to the aforementioned 4 or 5 genes. In other words, the transgene includes at least one of the aforementioned 4 or 5 genes, and other genes (which preferably do not inhibit presentation of the stratified squamous epithelium-forming ability and the skin appendage-inducing ability) may be further added.

Cells induced into the skin appendage-inducible epithelial cells can be selected using indicators whether there is a proliferation potential under a culture condition suitable for isolation and proliferation of keratinocytes, and whether the cells have characteristics of epithelial cells, as indicators. Specifically, such skin appendage-inducible epithelial cells exhibit a relatively high proliferation potential by being cultured on feeder cells (3T3-J2 feeder cells, 3T3 cells, mouse embryonic fibroblasts, human skin fibroblasts, or the like, of which the proliferation potential has been deactivated by mitomycin C or radiation treatment) suitable for isolation and proliferation of keratinocytes, or on serum-free keratinocyte medium, and therefore the skin appendage-inducible epithelial cells can be selected by continuing passage of the cells. It is also effective to add Rho kinase inhibitor (e.g., Y27632) which can relatively increase the number of keratinocyte divisions on a feeder. Also, the purity of the skin appendage-inducible epithelial cells can be increased by cell separation with a flow cytometry or a magnetic cell separator using epithelial cell-specific surface antigens (CDH1, Epi-CAM, etc.). If a reporter gene construct prepared by bonding a drug-resistant gene to a promoter for an epithelial cell marker gene (CDH1, Epi-CAM, etc.) has been previously transfected into somatic cells, cells that have acquired epithelial cell characteristics can grow in the presence of drugs, Therefore, cells induced into the skin appendage-inducible epithelial cells can be selected using the growth in the presence of drugs as an indicator.

The induced skin appendage-inducible epithelial cells obtained as described above can proliferate by being cultured in a liquid medium prepared by adding Rho kinase inhibitor on a feeder, and can stably proliferate while maintaining their skin appendage-inducing ability up to about the 10th passage common in the ordinary subculturing. For culturing the induced skin appendage-inducible epithelial cells, a medium typically used for culturing animal cells can be used. The suitable medium used for culturing skin appendage-inducible epithelial cells is exemplified by a serum-free keratinocyte medium (Keratinocyte-SFM, Life Technologies). It is also useful to add cytokines such as bFGF that accelerate proliferation of keratinocytes under a culture condition, and various pharmacologically active substances.

The induced skin appendage-inducible epithelial cells obtained as described above reconstruct and regenerate skin appendages by transplanting them in combination with skin appendage-inducible mesenchymal cells derived from neonatal animal skin into a chamber attached to a back of an immunodeficient animal. In contrast, the skin appendages can be neither reconstructed nor regenerated when adult-derived skin cells, or induced skin appendage-uninducible epithelial cells (e.g., cells capable of forming stratified squamous epithelium, which are induced by transfecting DNP63A gene, GRHL2 gene, TFAP2A gene, and cMYC gene induced by the method described in Non-Patent Document 15 into somatic cells) are transplanted in combination with skin appendage-inducible mesenchymal cells derived from neonatal animal skin, into a chamber attached to a back of an immunodeficient animal.

Similarly, neonatal animal-derived epithelial cells or cells isolated from adult-derived skin appendages can also reconstruct and regenerate the skin appendages by transplanting them in combination with skin appendage-inducible mesenchymal cells derived from neonatal animal skin. However, skin appendage-inducible mesenchymal cells are often contained in these cells isolated from animals. In other words, it has been conventionally difficult to separate and isolate only skin appendage-inducible epithelial cells from skin appendage-inducible mesenchymal cells. In contrast, the induced skin appendage-inducible epithelial cells obtained as described above do not contain skin appendage-inducible mesenchymal cells while having a skin appendage-inducing ability. On the basis of this property, transplantation of induced skin appendage-inducible epithelial cells in combination with mesenchymal cells unknown regarding the presence of the skin appendage-inducing ability can be used as an evaluation system for determining whether the mesenchymal cells have a skin appendage-inducing ability.

The induced skin appendage-inducible epithelial cells obtained as described above provides a therapeutic method for regeneration and neogenesis of skin and skin appendages by transplanting them into a patient in a pathological condition quantitatively or qualitatively lacking in skin appendages, such as a pathological condition of skin and skin appendage defect like a skin ulcer surface, a condition of hypoactive skin appendage and functions thereof due to age-related changes, and a condition after healing from skin ulcer.

In Non-Patent Document 15, a gene that induces cells capable of forming a stratified squamous epithelium is transfected into somatic cells in a living body to induce cells capable of forming stratified squamous epitheliums from the in vivo somatic cells incapable of forming stratified squamous epitheliums. In such a way, it is also possible to induce skin appendage-inducible epithelial cells from in vivo somatic cells by transfecting a transgene into the somatic cells in a living body using the aforementioned gene transfection means or vector.

In other words, the gene transfection means or vector for transfecting a gene that induces skin appendage-inducible epithelial cells into somatic cells in a living body provides a therapeutic method for regeneration and neogenesis of skin and skin appendages by transfecting the gene into a patient in a pathological condition lacking in skin appendages, such as a pathological condition of skin and skin appendage defect like a skin ulcer surface, a condition of hypoactive skin appendage and functions thereof due to age-related changes, and a condition after healing from skin ulcer to induce skin appendage-inducible epithelial cells.

### [Induced Mesenchymal Cell]

It is preferable that the method for producing the skin appendage-inducible cells, particularly mesenchymal cells according to the present invention includes a step of transfecting at least one gene selected from (1) PRDM1 gene, (2) FOXD1 gene, (3) ETV1 gene, (4) LEF1 gene, and (5) SHH gene into the skin appendage-uninducible somatic cells.

It is preferable that the method for producing the skin appendage-inducible cells, particularly mesenchymal cells according to the present invention includes a step of transfecting at least SHH gene into the skin appendage-uninducible somatic cells.

It is preferable that the method for producing the skin appendage-inducible cells, particularly mesenchymal cells according to the present invention includes a step of transfecting at least SHH gene and LEF1 gene into the skin appendage-uninducible somatic cells.

It is also preferable that the method for producing the skin appendage-inducible cells, particularly mesenchymal cells according to the present invention includes a step of transfecting at least one gene selected from (1) PRDM1 gene, (2) FOXD1 gene, and (3) ETV1 gene into the skin appendage-uninducible somatic cells.

It is also preferable that the method for producing the skin appendage-inducible cells, particularly mesenchymal cells according to the present invention includes a step of transfecting (1) SHH gene and (2) one or more genes selected from ETV1 gene, PRDM1 gene, FOXD1 gene, and LEF1 gene, into the skin appendage-uninducible somatic cells.

It is also preferable that the method for producing the skin appendage-inducible cells, particularly mesenchymal cells according to the present invention includes a step of transfecting at least one gene selected from (1) SHH gene, or SHH gene and LEF1 gene, and (2) one or more genes selected from ETV1 gene, PRDM1 gene, and FOXD1 gene, into the skin appendage-uninducible somatic cells.

It is also preferable that the method for producing the skin appendage-inducible cells, particularly mesenchymal cells according to the present invention includes a step of transfecting (1) ETV1 gene and PRDM1 gene; (2) FOXD1 gene and PRDM1 gene; or (3) ETV1 gene, PRDM1 gene, and FOXD1 gene, into the skin appendage-uninducible somatic cells.

As confirmed in the method for inducing the stratified squamous epithelium-forming ability in Non-Patent Document 15, the skin appendage-inducible mesenchymal cells can also be produced by adding other genes in addition to the aforementioned genes.

The induced skin appendage-inducible mesenchymal cells obtained as described above can be cultured e.g., in a DMEM medium supplemented with 10% FBS (fetal bovine serum), an Advanced-DMEM medium supplemented with N2-supplement, or the like. Also, it is useful to add cytokines such as bFGF that accelerate proliferation of mesenchymal cells under a culture condition, and various pharmacologically active substances.

The induced skin appendage-inducible mesenchymal cells obtained as described above (e.g., induced mesenchymal cells prepared by transfecting PRDM1 gene and FOXD1 gene into skin appendage-uninducible mesenchymal cells, and induced mesenchymal cells transfected with LEF1 gene and SHH gene into skin appendage-uninducible mesenchymal cells) can reconstruct and regenerate the skin appendages by transplanting them in combination with the skin appendage-inducible epithelial cells into a chamber attached to a back of an immunodeficient animal. In contrast, the skin appendages can be neither reconstructed nor regenerated when adult-derived skin mesenchymal cells are transplanted in combination with skin appendage-inducible epithelial cells into a chamber attached to a back of an immunodeficient animal.

The induced skin appendage-inducible mesenchymal cells obtained as described above can reconstruct and regenerate the skin appendages by transplanting them in combination with skin appendage-inducible cell epithelial cells derived from neonatal animal skin into a chamber attached to a back of an immunodeficient animal. Similarly, mesenchymal cells early isolated from a neonatal animal primary culture and cells isolated from adult-derived skin appendages can also reconstruct and regenerate the skin appendages by transplanting them in combination with skin appendage-inducible epithelial cells. However, skin appendage-inducible epithelial cells may be contained in these cells isolated from animals. In contrast, the induced skin appendage-inducible mesenchymal cells obtained according to the invention do not contain skin appendage-inducible epithelial cells while having a skin appendage-inducing ability. On the basis of this property, transplantation of induced skin appendage-inducible mesenchymal cells in combination with epithelial cells unknown regarding the presence of the skin appendage-inducing ability can be used as an evaluation system for determining whether the epithelial cells have a skin appendage-inducing ability.

The induced skin appendage-inducible mesenchymal cells obtained as described above provides a therapeutic method for regeneration and neogenesis of skin and skin appendages by transplanting them into a patient in a pathological condition quantitatively or qualitatively lacking in skin appendages, such as a pathological condition of skin and skin appendage defect like a skin ulcer surface, a condition of hypoactive skin appendage and functions thereof due to age-related changes, and a condition after healing from skin ulcer.

In Non-Patent Document 15, genes that induce cells capable of forming a stratified squamous epithelium are transfected into somatic cells in a living body to induce cells capable of forming stratified squamous epitheliums from the in vivo somatic cells incapable of forming stratified squamous epitheliums. In such a way, it is also possible to induce skin appendage-inducible mesenchymal cells from in vivo somatic cells by transfecting a transgene into the somatic cells in a living body using the aforementioned gene transfection means or vector.

In other words, the gene transfection means or vector for transfecting a gene that induces skin appendage-inducible mesenchymal cells into somatic cells in a living body provides a therapeutic method for regeneration and neogenesis of skins or skin appendages by transfecting the gene into a patient in a pathological condition lacking in skin appendages, such as a pathological condition of skin and skin appendage defect like a skin ulcer surface, a condition of hypoactive skin appendage and functions thereof due to age-related changes, and a condition after healing from skin ulcer to induce skin appendage-inducible mesenchymal cells. Therefore, the gene transfection means or vector for transfecting a gene that induces skin appendage-inducible mesenchymal cells serves as a composition for prepare the therapeutic preparations.

As described above, the skin appendage-inducible epithelial and mesenchymal cells obtained in the present invention have a proliferative ability and can regenerate skin appendages in vivo. Thus, the skin appendage-inducible epithelial and mesenchymal cells are effective in treatment of skin ulcers or the like caused by burns, trauma, iatrogenic injury (e.g., after tumor excision), decubitus, diabetic ulcer, and skin ulcer due to peripheral circulatory failure, and can be used as cell preparations (pharmaceutical composition) for regeneration of skin and skin appendage tissues.

When the skin appendage-inducible epithelial and mesenchymal cells are prepared as the cell preparations for regeneration of skin and skin appendage tissues, the preparations may contain, as necessary, a pharmaceutically acceptable dilution carrier together with the skin appendage-inducible epithelial and mesenchymal cells. Examples of the pharmaceutically acceptable dilution carrier include saline and buffer solution. Furthermore, the cell preparation may contain, as necessary, pharmacologically active ingredients, as well as ingredients as nutrient sources for the skin appendage-inducible epithelial and mesenchymal cells.

The skin appendage-inducible epithelial and mesenchymal cells may be applied to a skin disease site as tissue engineering preparations in which skin and skin appendage-like tissues were formed under a culture condition. The tissue engineering preparations contain a cell aggregate such as a sheet-like structure (e.g., epithelial cell sheet) and an organ-anlage with a three-dimensional structure.

When the skin appendage-inducible epithelial and mesenchymal cells are made into the tissue engineering preparations for regeneration of skin and skin appendage tissues, pharmacologically active ingredients, as well as ingredients as nutrient sources for the skin appendage-inducible epithelial and mesenchymal cells may be used as necessary together with the skin appendage-inducible epithelial and mesenchymal cells.

The skin appendage-inducible epithelial and mesenchymal cells may be formed into a cell aggregate such as a sheet-like structure, a skin- and skin appendage-like tissue with a three-dimensional structure, or an organ-anlage by using, as a scaffold, an extracellular matrix, e.g., collagen containing mesenchymal cells such as skin fibroblasts and adipose tissue-derived stromal cells, and thereafter may be applied to the skin disease site.

When the skin appendage-inducible epithelial and mesenchymal cells are made into the skin tissue and skin appendage-like tissue with a three-dimensional structure for regeneration of skin and skin appendages, pharmacologically active ingredients, as well as ingredients as nutrient sources for the skin appendage-inducible epithelial and mesenchymal cells may be used as necessary together with the skin appendage-inducible epithelial and mesenchymal cells. Use of scaffolding materials in this manner allows for more rapid regeneration of skin and skin appendages at the transplant site.

Usable scaffold materials are not limited as long as they are pharmacologically acceptable and selected as appropriate depending on a site of a cartilage tissue to which the preparation is applied. Examples of the scaffold materials include biocompatible gel materials. Preferable examples of the usable scaffold materials include collagen, fibronectin, hyaluronic acid, Matrigel, and a composite thereof. Each of these scaffold materials may be used alone or in combination of two or more types.

Also, a shape of the scaffold material is not limited and may be designed as appropriate depending on a shape of an injured site of skin and skin appendage tissues, to which the cell preparation is applied.

The method for applying the cell preparation to a disease site of an epithelial tissue is set as appropriate depending on a type of the cell preparation, a site on a skin tissue to which the preparation is applied, or the like. Examples of the method include a method of directly applying the cell preparation to a site of skin ulcer to be treated, a method of directly applying a tissue-like three-dimensional structure constructed under a culture condition, and a method of fixing a sheet or a three-dimensional structure by suture according to a skin grafting technique.

A dosage of the cell preparation applied to a disease site of a skin tissue should be set in an amount effective for regeneration of skin and skin appendage tissues as appropriate depending on a type of the cell preparation, a location of the epithelial tissue, a degree of the symptom, patient's age and gender, and the like.

A non-human mammal that has skin and skin appendage tissues formed from cells having induced skins and skin appendages by administration of the skin appendage-inducible epithelial and mesenchymal cells can be used as a tool for evaluating and analyzing a drug efficacy of a test substance on skin and/or skin appendage tissues. That means, a test substance is administered to a non-human mammal that has skin and skin appendage tissues formed from the skin appendage-inducible epithelial and mesenchymal cells to determine and analyze a drug efficacy of the test substance on the skin and/or skin appendage tissues, so that the drug efficacy of the test substance on the skin and/or skin appendage tissues can be evaluated and analyzed. Herein, the test substance refers to a substance to be evaluated and analyzed for its drug efficacy on skin and/or skin appendage tissues. Specific examples include candidate substances for a therapeutic drug for a skin and/or skin appendage disease. As the non-human mammal, mouse, rat, hamster, rabbit, cat, dog, sheep, pig, cow, goat, monkey, or the like is selected as appropriate.

A non-human mammal having skin and skin appendage tissues formed from the skin appendage-inducible epithelial and mesenchymal cells can be used as a model for examining an influence of external factors that damage the skin and/or skin appendage tissues, such as anticancer drugs and radiation, on the skin and/or skin appendage tissues.

The three-dimensional structure prepared by a scaffold such as a collagen gel containing the skin appendage-inducible epithelial and mesenchymal cells can be used as a tool for evaluating and analyzing a drug efficacy of a test substance on skin and/or skin appendage tissues. In other words, a test substance is administered to a three-dimensional structure prepared by a scaffold such as a collagen gel containing the skin appendage-inducible epithelial and mesenchymal cells and skin fibroblasts to determine and analyze the drug efficacy of the test substance on the skin and/or skin appendage tissues, so that the drug efficacy of the test substance on the skin and/or skin appendage tissues can be evaluated and analyzed. Herein, the test substance refers to a substance to be evaluated and analyzed for its drug efficacy on the skin and skin appendage tissues, and specific examples thereof include candidate substances for a skin and/or skin appendage disease therapeutic agent. In particular, skin appendage-inducible epithelial and mesenchymal cells induced from somatic cells that can be collected with relatively less invasiveness, such as peripheral circulating blood monocytes can be used to widely evaluate and analyze the drug efficacy on many donors with diverse genetic backgrounds.

The skin appendage-inducible epithelial and mesenchymal cells can be used as a tool for elucidating and analyzing various pathological conditions of skin and skin appendage tissues, and furthermore skin appendage-inducible epithelial and mesenchymal cells induced from human somatic cells are also useful as a tool for drug discovery and drug development for skin and skin appendage diseases. For example, skin appendage-inducible epithelial and mesenchymal cells are produced by transducing a transgene into human somatic cells, and furthermore, these cells are cultured to prepare skin and skin appendage-like tissues, a test substance is administered to the prepared skin and skin appendage-like tissues. Thereby, a drug efficacy of the test substance on the skin and skin appendage-like tissues can be evaluated and analyzed, or stresses on the skin and skin appendage-like tissues can be evaluated and analyzed by loading the tissues with stresses such as an anticancer and a radiation. The drug efficacy or stresses may be evaluated and analyzed e.g., by comparison between tissues to which the test substance is administered or tissues loaded with a stress and tissues free from the test substance or the stresses.

### 2. EXPERIMENTAL RESULT

The present invention will be described below in detail based on Examples and the like, but the present invention is not limited to Examples and the like.

### [Reference Example 1] Transplantation Experiment of Adult Mouse Skin-Derived Epithelial Cell and Adult Mouse Skin-Derived Mesenchymal Cell

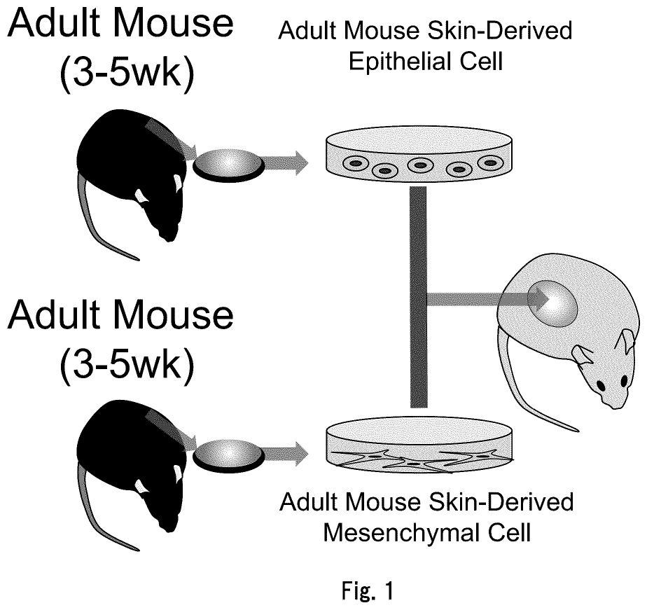

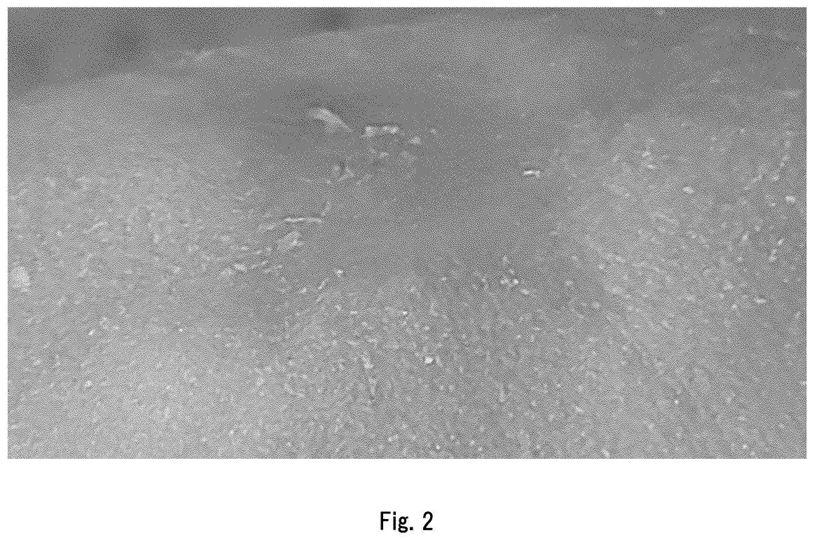

A skin specimen obtained from a back of an adult mouse was treated with 0.25% trypsin at 4° C. overnight, and cells collected from an epidermal tissue peeled off from a dermis and a surface layer of a dermis were seeded on 3T3-J2 feeder cells in a keratinocyte F medium containing Y27632 (manufactured by SELLECK) as Rho-kinase inhibitor so as to collect primary cultured epithelial cells derived from an adult mouse skin. On the other hand, a skin specimen obtained from a waist-back site of an adult mouse was treated with 0.25% trypsin at 4° C. overnight, and a dermis tissue from which an epidermal tissue was peeled off or a subcutaneous adipose tissue was treated with 0.1% collagenase at 37° C. for 1 hour, and collected cells were seeded on a DMEM medium supplemented with 10% fetal bovine serum so as to collect primary cultured mesenchymal cells derived from an adult mouse skin. The primary cultured adult mouse skin-derived epithelial cells and the primary cultured adult mouse skin-derived mesenchymal cells cultured for 1 to 3 days were transplanted into a silicone chamber attached to a back of an immunodeficient animal. In accordance with a conventional method of a skin appendage reconstruction assay using a silicone chamber, one week after the transplantation, a hole was formed on the top of the silicone chamber, and 2 weeks after the transplantation, the silicone chamber was removed, and observation was continued for 4 to 5 weeks after the transplantation (FIG. 1). The chamber site was epithelialized by the transplanted cells, but the skin appendages could not be reconstructed and regenerated (FIG. 2).

The 3T3-J2 feeder cells used as a feeder are derived from a cell line distributed from Japan Tissue Engineering Co., Ltd. The 3T3-J2 feeder cells were retained in a 3T3 cell medium prepared by adding 10% fetal bovine serum medium to a DMEM medium according to a routine method and then treated in medium with a 10 μg/ml mitomycin C for 1 hour at a day before use as feeder cells. Then, the cells were passaged at a concentration of 2.0×10⁵ cells/well and used as the feeder.

### [Reference Example 2] Transplantation Experiment of Neonatal Mouse Skin-Derived Epithelial Cell and Neonatal Mouse Skin-Derived Mesenchymal Cell

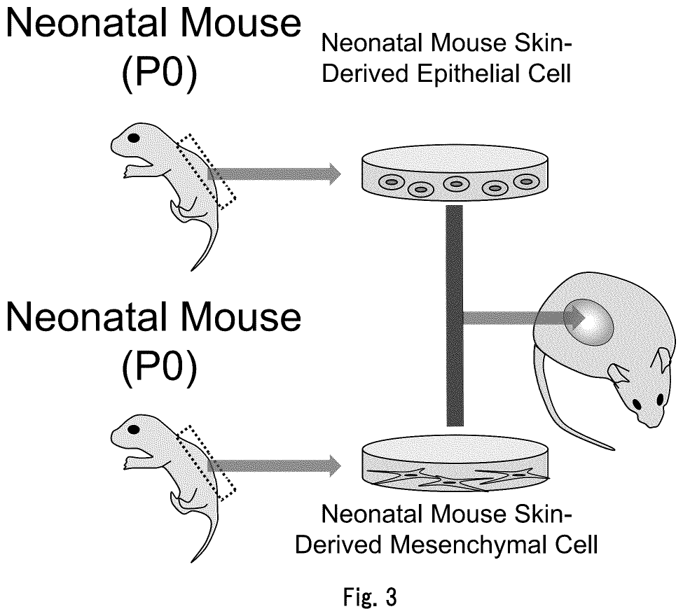

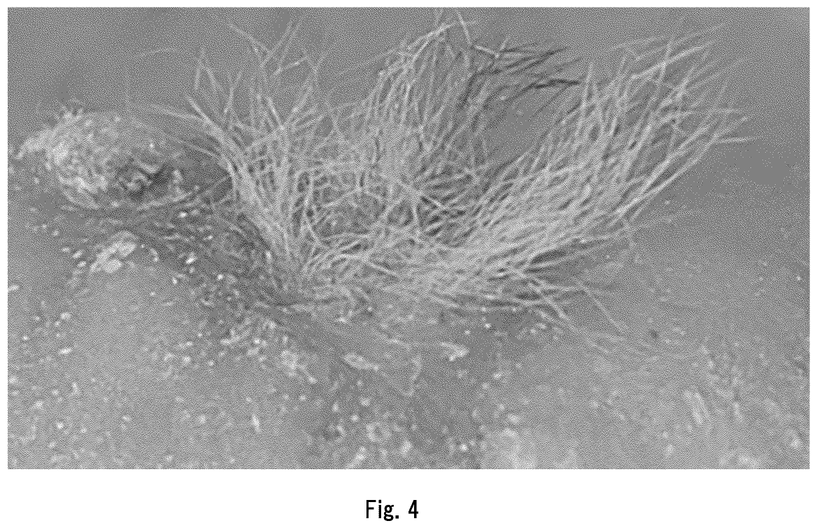

A skin specimen obtained from a back of a neonatal mouse was treated with 0.25% trypsin at 4° C. overnight, and cells collected from an epidermal tissue peeled off from a dermis and a surface layer of a dermis were seeded on 3T3-J2 feeder cells in a keratinocyte F medium containing Y27632 (manufactured by SELLECK) as Rho-kinase inhibitor so as to collect primary cultured epithelial cells derived from a neonatal mouse skin. On the other hand, a skin specimen obtained from a back of a neonatal mouse was treated with 0.25% trypsin at 4° C. overnight, and a dermis tissue from which an epidermal tissue was peeled off and a subcutaneous adipose tissue were treated with 0.1% collagenase at 37° C. for 1 hour, and collected cells were seeded on a DMEM medium supplemented with 10% fetal bovine serum so as to collect primary cultured mesenchymal cells derived from a neonatal mouse skin. The primary cultured neonatal mouse skin-derived epithelial cells and the primary cultured neonatal mouse skin tissue-derived mesenchymal cells cultured for 1 to 3 days were transplanted into a silicone chamber attached to a back of an immunodeficient animal. One week after the transplantation, a hole was formed on the top of the silicone chamber, and 2 weeks after the transplantation, the silicone chamber was removed, and observation was continued for 4 to 5 weeks after the transplantation (FIG. 3). Hair growth, as well as reorganization and regeneration of skin appendages were obtained on the chamber site (FIG. 4).

### [Comparative Example 1] Transplantation Experiment of Cell that is Capable of Forming Stratified Squamous Epitheliums and Neonatal Mouse Skin-Derived Mesenchymal Cell

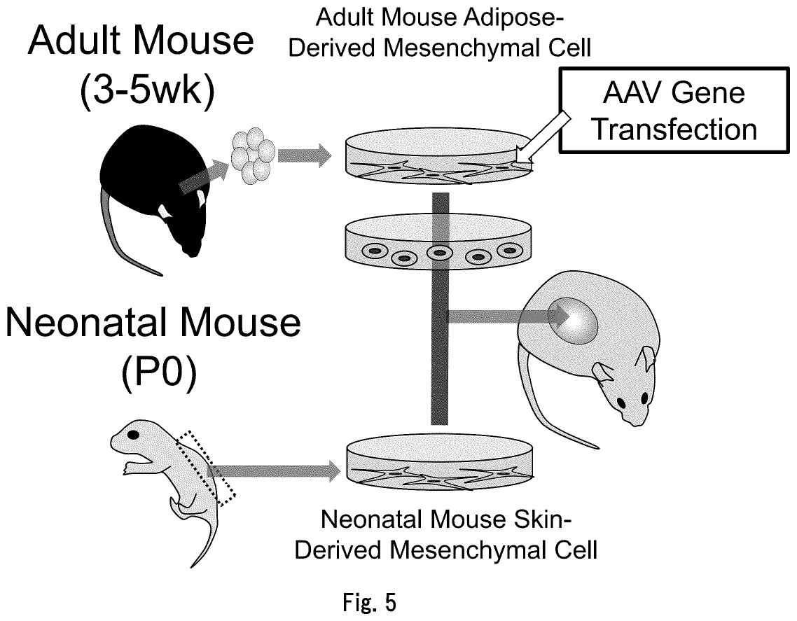

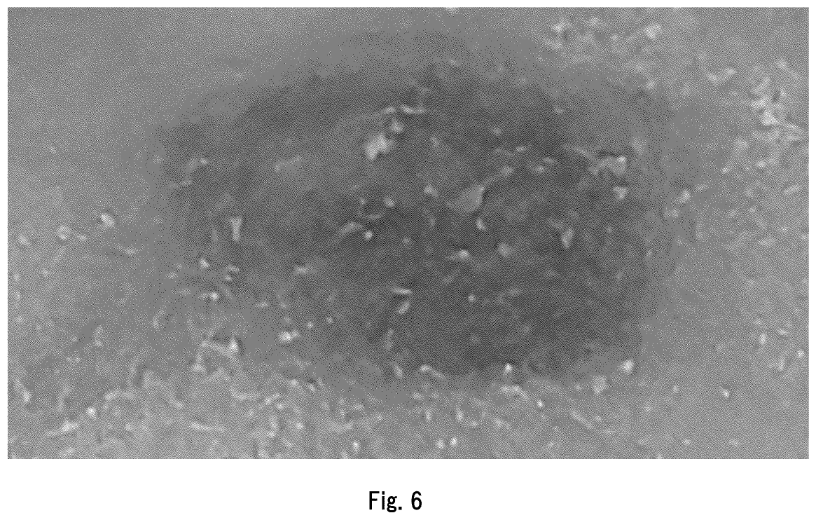

An adult mouse subcutaneous adipose tissue was treated with 0.1% collagenase at 37° C. for 1 hour, and collected cells were seeded on a DMEM medium supplemented with 10% fetal bovine serum so as to collect primary cultured mesenchymal cells derived from an adult mouse skin. After subculture up to the second passage, DNP63A gene, GRHL2 gene, TFAP2A gene, and c-MYC gene were transfected by using AAVs so as to create induced epithelial cells with the ability to form stratified squamous epithelium. The aforementioned induced epithelial cells and the neonatal mouse skin tissue-derived mesenchymal cells were transplanted into a silicone chamber attached to a back of an immunodeficient animal. One week after the transplantation, a hole was formed on the top of the silicone chamber, and 2 weeks after the transplantation, the silicone chamber was removed, and observation was continued for 4 to 5 weeks after the transplantation (FIG. 5). The chamber site was epithelialized by the transplanted cells, but the skin appendages could not be reconstructed and regenerated (FIG. 6).

### Comparative Example 2

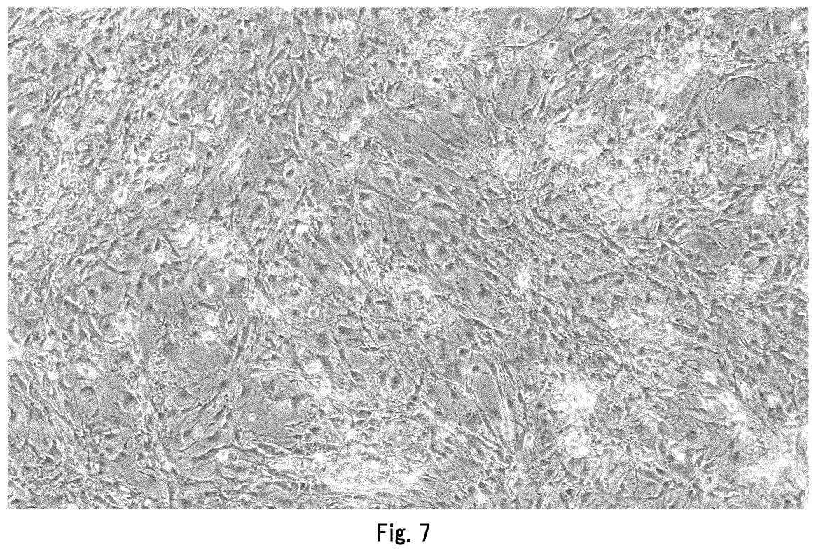

To develop a method for inducing skin appendage-inducible epithelial cells from primary cultured adult mouse adipose-derived mesenchymal cells by gene transfection, the primary cultured adult mouse adipose-derived mesenchymal cells were subcultured up to the second passage, and then DNP63A gene, GRHL2 gene, TFAP2A gene, c-MYC gene, and SHH genes were transfected into the cells using AAVs. However, induced epithelial cells could not be obtained (FIG. 7).

### Comparative Example 3

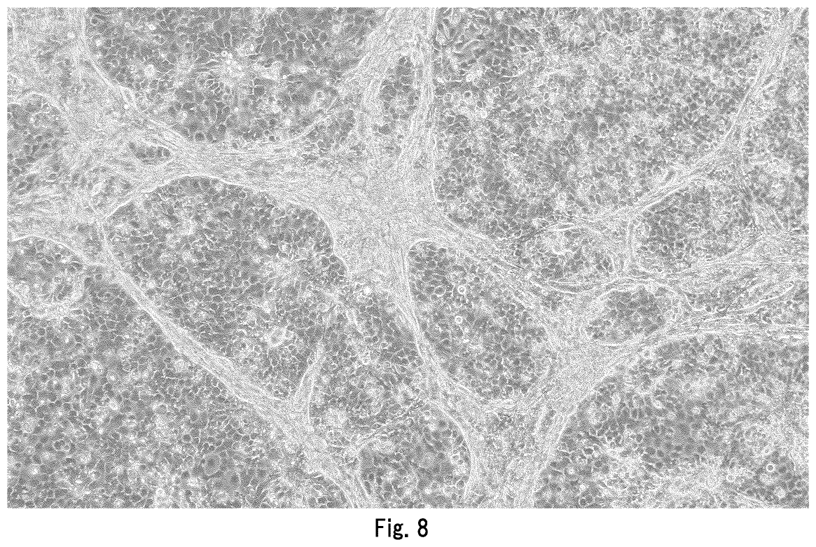

To develop a method for inducing skin appendage-inducible epithelial cells from primary cultured adult mouse adipose-derived mesenchymal cells by gene transfection, the primary cultured adult mouse adipose-derived mesenchymal cells were subcultured up to the second passage, and then DNP63A gene, GRHL2 gene, TFAP2A gene, c-MYC gene, and FOXD1 genes were transfected into the cells using AAVs. As a result, induced epithelial cells were obtained (FIG. 8).

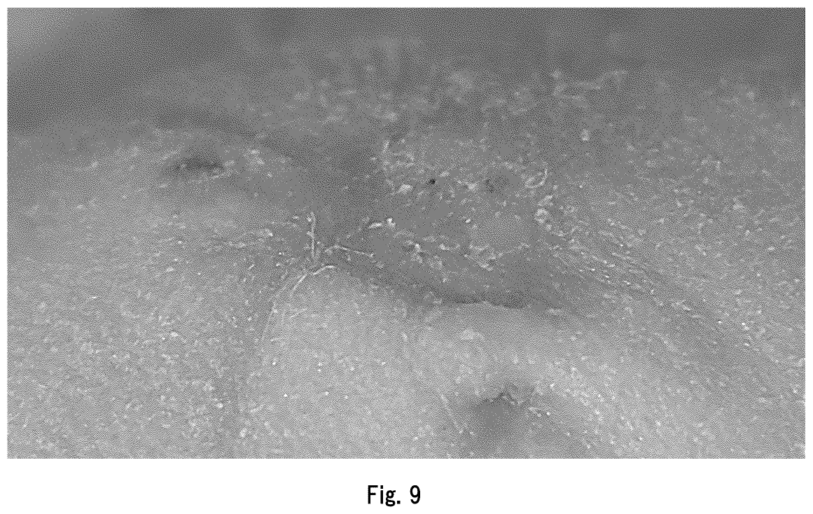

The induced epithelial cells obtained as described above and the primary cultured neonatal mouse-derived mesenchymal cells were transplanted into a silicone chamber attached to a back of an immunodeficient animal. One week after the transplantation, a hole was formed on the top of the silicone chamber, and 2 weeks after the transplantation, the silicone chamber was removed, and observation was continued for 4 to 5 weeks after the transplantation. The chamber site was epithelialized by the transplanted cells, but the skin appendages could not be reconstructed and regenerated (FIG. 9).

### [Example 1] Transplantation Experiment of Induced Skin Appendage-Inducible Epithelial Cell and Neonatal Mouse Skin-Derived Mesenchymal Cell

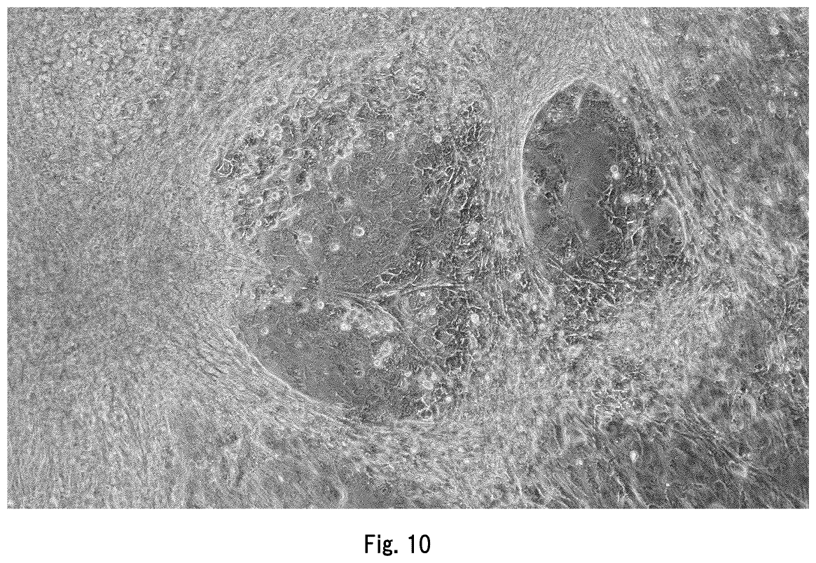

To develop a method for inducing skin appendage-inducible epithelial cells from primary cultured adult mouse adipose-derived mesenchymal cells by gene transfection, the primary cultured adult mouse adipose-derived mesenchymal cells were subcultured up to the second passage, and then DNP63A gene, GRHL2 gene, TFAP2A gene, c-MYC gene, and LEF1 genes were transfected into the cells using AAVs. As a result, induced epithelial cells were obtained (FIG. 10).

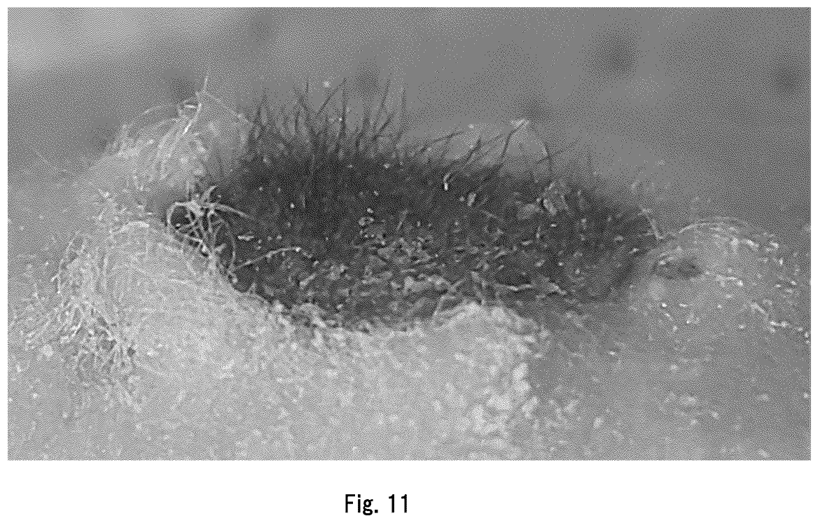

The induced epithelial cells obtained as described above and the primary cultured neonatal mouse-derived mesenchymal cells were transplanted into a silicone chamber attached to a back of an immunodeficient animal. One week after the transplantation, a hole was formed on the top of the silicone chamber, and 2 weeks after the transplantation, the silicone chamber was removed, and observation was continued for 4 to 5 weeks after the transplantation. Hair growth, as well as reorganization and regeneration of skin appendages were obtained on the chamber site (FIG. 11). The induced epithelial cells obtained as described above fulfilled the characteristics of induced skin appendage-inducible epithelial cells. Details will be described in Example 1 below.

### [Comparative Example 4] Transplantation Experiment of Neonatal Mouse Skin-Derived Epithelial Cell and Induced Mesenchymal Cell

Genes that were relatively strongly expressed in skin appendage-inducible mesenchymal cells (SOX2 gene, LEF1 gene, HOXA9 gene, HOXC4 gene, HOXC9 gene, HOXC13 gene, JARID2 gene, HEY1 gene, HEY2 gene, FOXO1 gene, FOXD1 gene, EGR3 gene, MEF2C gene, LHX2 gene, PRRX1 gene, CREB3 gene, ETV1 gene, TFAP2A gene, cMYC gene, TBX6 gene, MSX2 gene, SHH gene, PRDM1 gene) were selected as candidate genes for inducing skin appendage-inducible mesenchymal cells that reconstructed and regenerated skin appendages by transplantation in combination with skin appendage-inducible epithelial cells, and that had been induced by transfecting a gene into primary cultured adult mouse adipose-derived mesenchymal cells.

The primary cultured neonatal mouse skin-derived epithelial cells were considered as skin appendage-inducible epithelial cells. Then, the epithelial cells after the second or third passage of subculture for removing neonatal mouse skin-derived mesenchymal cells that had been contained during the primary culture were transplanted into a silicone chamber attached to a back of an immunodeficient animal together with cells prepared by subculturing the primary cultured adult mouse adipose-derived mesenchymal cells up to the second passage and then transfected with a combination of multiple genes that were relatively strongly expressed in skin appendage-inducible mesenchymal cells using a retroviral vector. Some mice showed a small amount of hair growth as well as reconstruction and regeneration of skin appendages on the chamber site, but no consistent tendencies according to transgenes were observed. Thus, the epithelial cells obtained by subculturing primary cultured neonatal mouse skin-derived epithelial cells up to the second or third passage in order to remove the neonatal mouse skin-derived mesenchymal cells were highly likely to actually contain the neonatal mouse skin-derived mesenchymal cells. It was difficult to obtain epithelial cells without containing the neonatal mouse skin-derived mesenchymal cells while maintaining the skin appendage-inducing ability of the primary cultured neonatal mouse skin-derived epithelial cells.

In this regard, the induced epithelial cells obtained by transfecting DNP63A gene, GRHL2 gene, TFAP2A gene, c-MYC gene, and LEF1 gene (Examples 1 and 2), or DNP63A gene, GRHL2 gene, TFAP2A gene, and LEF1 gene (Example 3) into the primary cultured adult mouse adipose-derived mesenchymal cells do not contain the skin appendage-inducible mesenchymal cells while having the skin appendage-inducing ability. This property could be used for evaluation to determine whether certain mesenchymal cells have the skin appendage-inducing ability by transplantation in combination with the induced skin appendage-inducible epithelial cells as described in Examples 2 and 3.

### Example 2

### Transplantation Experiment of Induced Skin Appendage-Inducible Epithelial Cell and Induced Skin Appendage-Inducible Mesenchymal Cell

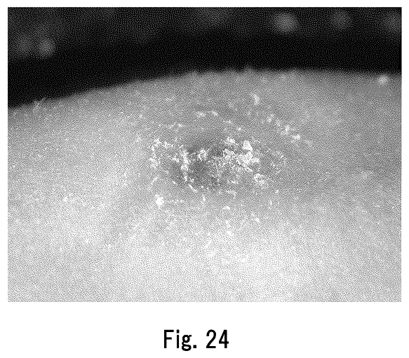

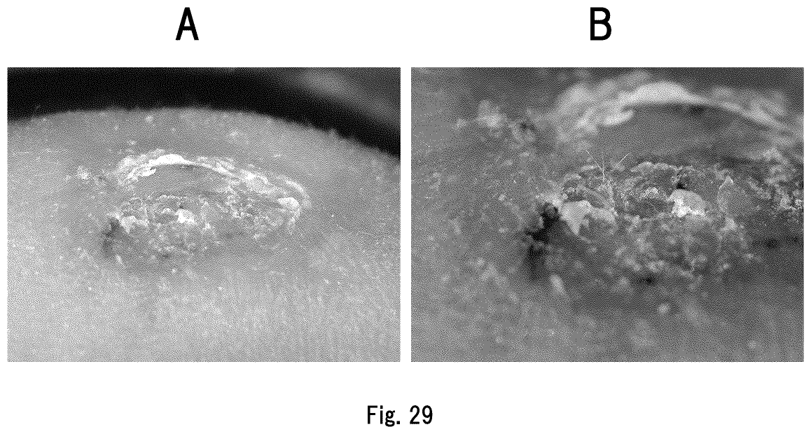

First, SHH gene and LEF1 gene, which have been suggested to be important as environmental factors for regeneration of hair follicle by researches using gene-recombinant animals, were selected as candidates. Induced skin appendage-inducible epithelial cells prepared by subculturing primary cultured adult mouse adipose-derived mesenchymal cells up to the second passage and then transfected with DNP63A gene, GRHL2 gene, TFAP2A gene, c-MYC gene, and LEF1 gene using AAVs (Example 1) was transplanted in combination, into a chamber attached to a back of an immunodeficient animal in combination with mesenchymal cells prepared by subculturing primary cultured adult mouse adipose-derived mesenchymal cells up to the second passage and then transfected with SHH gene alone using a retrovirus vector (Example 2-13), or mesenchymal cells transfected with a combination of SHH gene and LEF1 gene (Example 2-8). One week after the transplantation, a hole was formed on the top of the silicone chamber, and 2 weeks after the transplantation, the silicone chamber was removed, and then observation was continued for 4 to 5 weeks after the transplantation. A few hairs were observed to grow on the chamber site (FIG. 24 and FIG. 29).

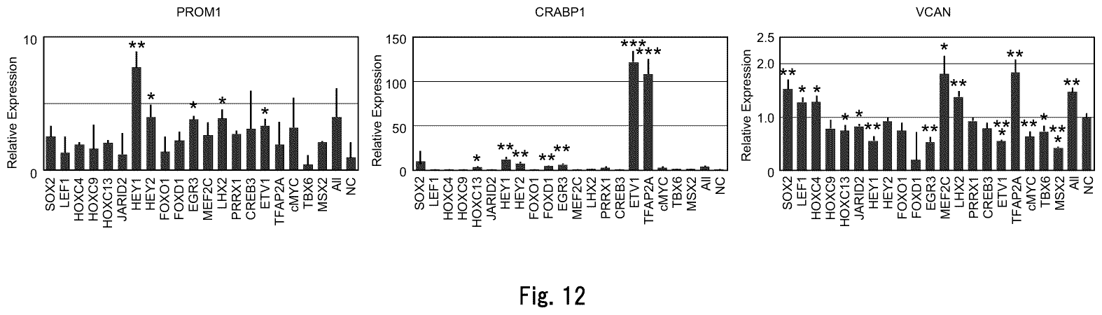

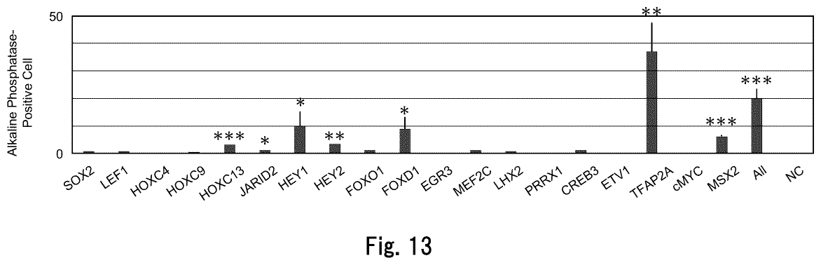

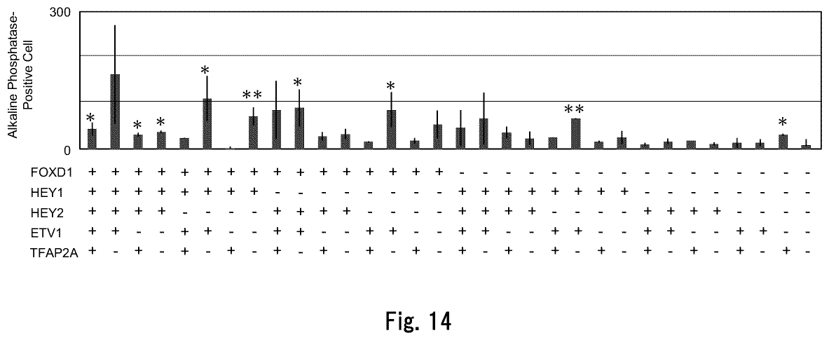

Next, a change in expression of marker genes (PROM1 gene, CRABP1 gene, and VCAN gene) for skin appendage-inducible mesenchymal cells (FIG. 12) and a change in alkaline phosphatase expression (FIG. 13) were examined when transfecting each candidate gene in order to further select factors for inducing skin appendage-inducible mesenchymal cells that reconstructed and regenerated skin appendages by transplantation in combination with induced skin appendage-inducible epithelial cells. FOXD1 gene, HEY1 gene, HEY2 gene, ETV1 gene, and TFAP2A gene were selected as highly probable candidate factors because they strongly express marker genes and alkaline phosphatase by single gene transfection. Among these candidate factors, a plurality of candidate genes were further transfected in combination, and at this time, a change in alkaline phosphatase expression (FIG. 14) was examined, and this change was used as a reference for selecting candidate genes. Throughout a series of analyses, ETV1 and FOXD1 were eventually considered to be probably effective as factors for inducing the induced skin appendage-inducible mesenchymal cells, and the effectiveness of these genes was confirmed in the subsequent in vivo transplantation experiment.

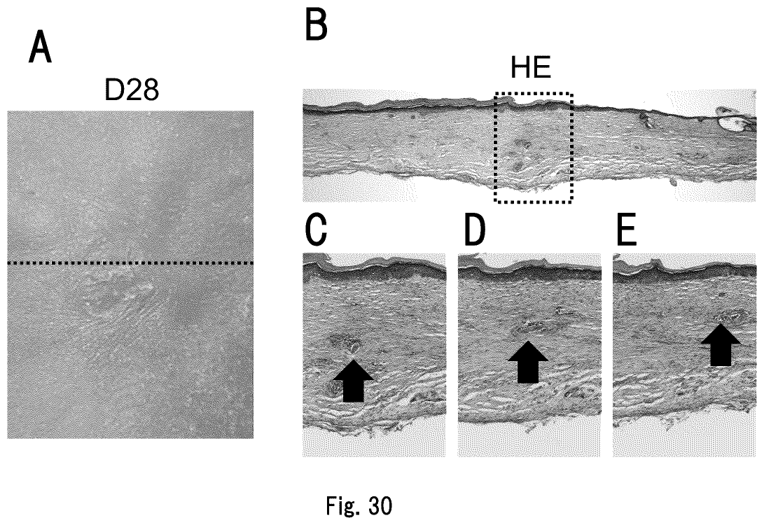

In order to select factors for inducing skin appendage-inducible mesenchymal cells with a mechanism different from those of SHH gene, LEF1 gene, and environmental factors, ETV1 gene, FOXD1 gene, and PRDM1 gene were selected as candidates. Induced skin appendage-inducible epithelial cells prepared by subculturing primary cultured adult mouse adipose-derived mesenchymal cells up to the second passage and then transfected with DNP63A gene, GRHL2 gene, TFAP2A gene, c-MYC gene, and LEF1 gene using AAVs (Example 1) was transplanted in combination, into a chamber attached to a back of an immunodeficient animal in combination with mesenchymal cells prepared by subculturing primary cultured adult mouse adipose-derived mesenchymal cells up to the second passage and then transfected with a combination of ETV1 gene, FOXD1 gene, and PRDM1 gene using a retrovirus vector (Example 2-14). One week after the transplantation, a hole was formed on the top of the silicone chamber, and 2 weeks after the transplantation, the silicone chamber was removed, and observation was continued for 4 to 5 weeks after the transplantation. Hair follicle-like structures and mature hair shafts were observed by a histological investigation on a central subcutaneous region of the cell transplantation site where hairs were not normally observed (FIG. 30).

It was considered that skin appendage-inducible mesenchymal cells could be induced with a mechanism different from those of SHH gene, LEF1 gene, and environmental factors by gene transfection of a combination of ETV1 gene, FOXD1 gene, and PRDM1 genes. This suggested that hair growth as well as reconstruction and regeneration of skin appendages could be achieved more efficiently by transplantation of multiple types of cells transfected with these genes.

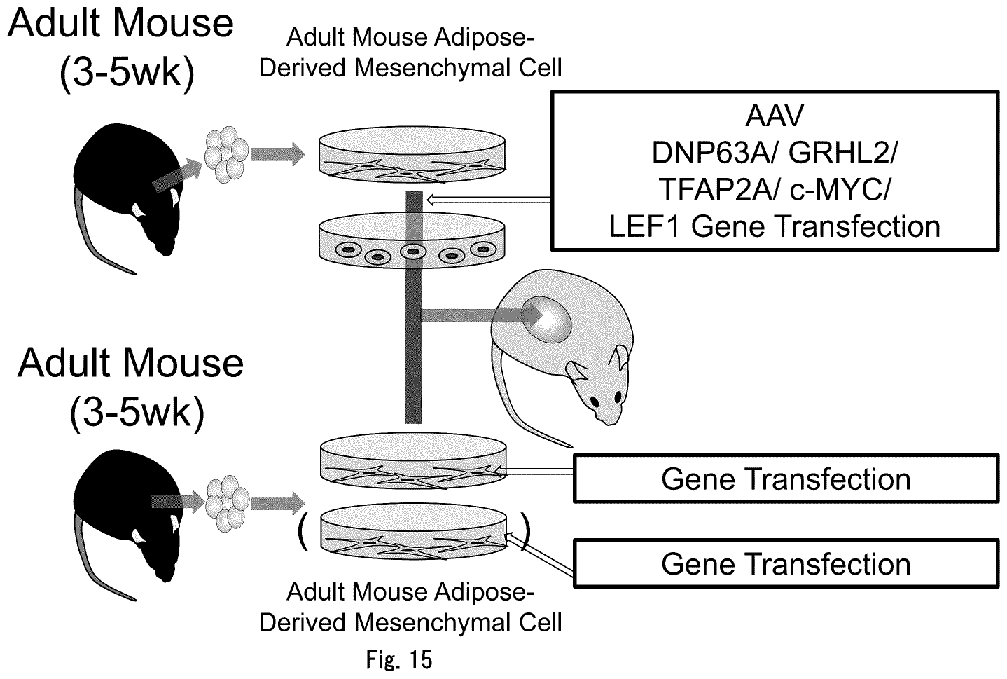

In order to select factors for inducing skin appendage-inducible mesenchymal cells that reconstruct and regenerate skin appendages by transplantation in combination with induced skin appendage-inducible epithelial cells, induced skin appendage-inducible cells prepared by subculturing primary cultured adult mouse adipose-derived mesenchymal cells up to the second passage and then transfected with DNP63A gene, GRHL2 gene, TFAP2A gene, c-MYC gene, and LEF1 gene using AAVs (Example 1) was transfected in combination, into a chamber attached to a back of an immunodeficient animal in combination with one or two types of mesenchymal cells prepared by subculturing primary cultured adult mouse adipose-derived mesenchymal cells up to the second passage and then transfected with a combination of multiple genes that were relatively strongly expressed in skin appendage-inducible mesenchymal cells using a retrovirus vector (FIG. 15). One week after the transplantation, a hole was formed on the top of the silicone chamber, and 2 weeks after the transplantation, the silicone chamber was removed, and observation was continued for 4 to 5 weeks after the transplantation. The genes transfected into the first and second induced mesenchymal cells are as listed in Table 2. In animals to which a combination of one or two types of induced mesenchymal cells transfected with genes in 13 combination patterns described in Examples 2-1 to 2-13 was transplanted, hair growth as well as reconstruction and regeneration of skin appendages were achieved on the chamber site.

TABLE 2

| Example | First Induced Mesenchymal Cell | | | | | Second Induced Mesenchymal Cell | | |
|---|---|---|---|---|---|---|---|---|
| 2-1 | SHH | LEF1 | | | | ETV1 | FOXD1 | PRDM1 |
| 2-2 | SHH | | | | | ETV1 | FOXD1 | PRDM1 |
| 2-3 | SHH | LEF1 | | | | ETV1 | FOXD1 | |
| 2-4 | SHH | LEF1 | | | | | FOXD1 | PRDM1 |
| 2-5 | SHH | LEF1 | | | | ETV1 | | PRDM1 |
| 2-6 | SHH | | ETV1 | FOXD1 | PRDM1 | | | |
| 2-7 | SHH | LEF1 | ETV1 | FOXD1 | PRDM1 | | | |
| 2-8 | SHH | LEF1 | | | | | | |
| 2-9 | SHH | LEF1 | | | | | | PRDM1 |
| 2-10 | SHH | | | | | | FOXD1 | PRDM1 |
| 2-11 | SHH | | | | | | | PRDM1 |
| 2-12 | SHH | | | | | | FOXD1 | |
| 2-13 | SHH | | | | | | | |
| 2-14 | | | ETV1 | FOXD1 | PRDM1 | | | |

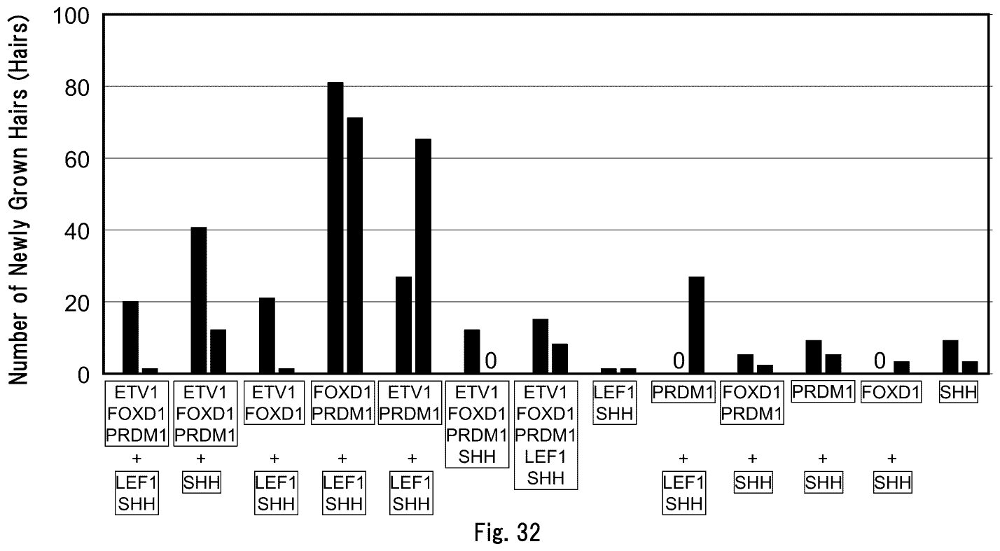

Each combination of the transgenes was subjected to two experiments, and the number of newly grown hairs was counted by stereomicroscopic investigation, and as a result, it was found that a larger number of hairs could be newly grown and skin appendages could be reconstructed and regenerated by combining transgenes and cells. FIG. 32 lists the results of counting the number of newly grown hairs in each of the two experiments in Examples 2-1 to 2-13. Also, it was found that, when using multiple types of transgenes in the same combination, a better result could be obtained by transplantation of multiple types of induced mesenchymal cells. For example, the transplantation of the first induced mesenchymal cells transfected with SHH gene and the second induced mesenchymal cells transfected with ETV1 gene, FOXD1 gene, and PRDM1 gene as in Example 2-2 showed a result superior to that of the transplantation of one cell transfected with ETV1 gene, FOXD1 gene, PRDM1 gene, and SHH gene as for the first induced mesenchymal cells in Example 2-6.

The induced mesenchymal cells obtained in this way were found to satisfy the characteristics of the skin appendage-inducible mesenchymal cells that reconstructed and regenerated skin appendages by transplantation in combination with induced skin appendage-inducible epithelial cells.

### Example 3

Furthermore, induced epithelial cells prepared by subculturing primary cultured adult mouse adipose-derived mesenchymal cells up to the second passage and then transfected with DNP63A gene, GRHL2 gene, TFAP2A gene, and LEF1 gene using AAV, mesenchymal cells prepared by subculturing primary cultured adult mouse adipose-derived mesenchymal cells up to the second passage and then transfected with ETV1 gene, FOXD1 gene, PRDM1 gene using a retrovirus vector, and mesenchymal cells transfected with LEF1 gene and SHH gene were transplanted in combination, into a chamber attached to a back of an immunodeficient animal (FIG. 15). One week after the transplantation, a hole was formed on the top of the silicone chamber, and 2 weeks after the transplantation, the silicone chamber was removed, and observation was continued for 4 to 5 weeks after the transplantation. Since hairs were grown and skin appendages were reconstructed and regenerated on the chamber site, the induced epithelial cells transfected with DNP63A gene, GRHL2 gene, TFAP2A gene, and LEF1 gene using AAV were found to satisfy the characteristics of the skin appendage-inducible epithelial cells. Although viral vectors were used for the gene transfection in Examples, the vector is not limited to viral vectors, and any vector that enhances expression of a transgene, such as plasmid vectors and mRNA vectors can be used for administration.

### Example 1

1-1 Induction of Induced Skin Appendage-Inducible Epithelial Cell from Primary Cultured Adult Mouse Adipose-Derived Mesenchymal Cell

AAVs those expressed DNP63A gene, GRHL2 gene, TFAP2A gene, c-MYC gene, and LEF1 gene for transfecting genes into adult mouse adipose-derived mesenchymal cells under a culture condition were prepared.

According to a method described in Reference Literature 2 (Grieger, J. C., Choi, V. W. & Samulski, R. J. Production and characterization of adeno-associated viral vectors. “Nat Protocol”, 2006, 1 (3), p. 1412-1428), the AAVs were prepared using: Gateway human entry clone purchased from NBRC (Biological Resource Center, National Institute of Technology and Evaluation), plasmids purchased from manufacturers (System Biosciences, LLC, GeneCopoeia, Inc., and OriGene Technologies, Inc.), pAAV plasmids prepared by cloning a coding sequences amplified by PCR using cDNAs prepared from mRNA collected from BJ fibroblast (ATCC) as a template by In-Fusion® HD Cloning Kit (Clontech Laboratories, Inc.) into a pAAV-CAG-GFP (Addgene Plasmid #37825) backbone, and AAV-DJ Rep-Cap Plasmid (Cell Biolabs Inc.). Titering of the AAVs were performed by qPCR according to a routine method.

A subcutaneous adipose specimen sampled from a waist-back site of an adult mouse (C57BL/6) was cut into small pieces and treated with 0.1% collagenase at 37° C. for 1 hour in order to collect cells. Then, the cells were seeded in a DMEM medium supplemented with 10% fetal bovine serum in order to collect primary cultured adult mouse adipose-derived mesenchymal cells. The mesenchymal cells were subcultured up to the second passage and then cryopreserved in a DMEM medium supplemented with 15% fetal bovine serum containing 10% DMSO. The frozen cells were thawed, subcultured up to the first passage, and then seeded in a DMEM medium supplemented with 10% fetal bovine serum at 50,000 to 100,000 cells per 1 well. On the next day, the cells were infected with 10¹⁰ Gene Copy (GC) DNP63A gene-expressing AAVDJ, 10⁹ GC GRHL2 gene-expressing AAVDJ, 5×10⁹ GC TFAP2A gene-expressing AAVDJ, 5×10⁹ GC cMYC gene-expressing AAVDJ, and 2×10⁹ GC LEF1 gene-expressing AAVDJ per 1 well. On day 2, day 3, and day 5, the medium was replaced with fresh medium. On day 6, the medium was replaced with a keratinocyte F medium containing Rho-kinase inhibitor Y27632 (FUJIFILM Wako Pure Chemical Corporation), and thereafter, the medium was replaced daily.

The keratinocyte F medium was prepared according to a routine method. In this study, the keratinocyte F medium was prepared by adding 25 ml of fetal bovine serum to a mixture of 225 ml of F12 medium (FUJIFILM Wako Pure Chemical Corporation) and 225 ml of DME/F12 medium (FUJIFILM Wako Pure Chemical Corporation), and adjusted so as to contain 24 μg/ml of adenine (Sigma-Aldrich Co. LLC), 8.4 ng/ml of cholera toxin (FUJIFILM Wako Pure Chemical Corporation), 5 μg/ml of insulin (FUJIFILM Wako Pure Chemical Corporation), and 0.4 μg/ml of hydrocortisone (Sigma-Aldrich Co. LLC), 100 U/ml of penicillin (FUJIFILM Wako Pure Chemical Corporation), 100 μg/ml of streptomycin (FUJIFILM Wako Pure Chemical Corporation), and 10 ng/ml of epidermal growth factor (EGF) (FUJIFILM Wako Pure Chemical Corporation). Furthermore, the keratinocyte F medium was adjusted so as to contain 10 μM of Y-27632 (Selleck Chemicals LLC) in this study.

From day 10 to day 15, appearance of epithelial-like colonies as presented in FIG. 10 was observed, and therefore, on day 15, the colonies were passaged on 3T3-J2 feeder cells prepared in a 6-well plate treated with mitomycin. After the passage, the medium was replaced every 2 days, and when the cells became a subconfluent state (in about a week), the cells were passaged at a dilution ratio of 1:3 to 1:8 to increase the number of cells. The cells were cryopreserved in a 10% DMSO-containing keratinocyte F medium supplemented with Y27632, and thawed and amplified as necessary for use in a transplantation experiment.

1-2 Regeneration of Skin Appendages Using Skin Appendage-Inducible Epithelial Cell Induced from Primary Cultured Adult Mouse Adipose-Derived Mesenchymal Cell, and Neonatal Mouse Skin-Derived Mesenchymal Cell

The induced epithelial cells obtained in Examples 1-1 and the primary cultured neonatal mouse skin tissue-derived mesenchymal cells were transplanted into a silicone chamber attached to a back of an immunodeficient animal according to the following procedure. The induced epithelial cells were cultured on 3T3-J2 feeder cells prepared on two 10 cm cell culture dishes (Violamo) using a keratinocyte F medium until the cells became a subconfluent state, then treated with 0.25% trypsin at 37° C. for 2 minutes, and the trypsin solution was aspirated to remove the feeder cells. Furthermore, the induced epithelial cells were treated with 0.25% trypsin at 37° C. for 5 to 10 minutes, and collected in 10 ml of keratinocyte F medium to prepare a suspension containing the induced epithelial cells. On the other hand, a skin specimen obtained from a back of a neonatal mouse was treated with 0.25% trypsin at 4° C. overnight, then dermis tissues from which epidermal tissue was peeled off and subcutaneous adipose tissues were treated with 0.1% collagenase at 37° C. for 1 hour, and collected cells were seeded on DMEM medium supplemented with 10% fetal bovine serum per 1 well of a 6-well plate (VIOLAMO) in order to obtain primary cultured neonatal mouse skin-derived mesenchymal cells. The cells were cultured for 3 days, treated with 0.25% trypsin at 37° C. for 5 to 10 minutes, and collected in a DMEM medium supplemented with 10% fetal bovine serum in order to prepare a primary cultured neonatal skin tissue-derived mesenchymal cell suspension. Both suspensions were each filtered using a 100 μm cell strainer, mixed, and centrifuged at 300 G for 5 minutes to obtain a cell pellet. This cell pellet was suspended in 100 μl of a medium of a 1:1 mixture of a keratinocyte F medium and a DMEM medium supplemented with 10% fetal bovine serum, and the suspension was transplanted into a dome-shaped silicone chamber attached to a back of an immunodeficient animal. One week after the transplantation, a hole was formed on the top of the silicone chamber, and 2 weeks after the transplantation, the silicone chamber was removed, and observation was continued for 4 to 5 weeks after the transplantation. Hair growth as well as reconstruction and regeneration of skin appendages were achieved on the chamber site (FIG. 11). The induced epithelial cells obtained as described above fulfilled the characteristics of induced skin appendage-inducible epithelial cells.

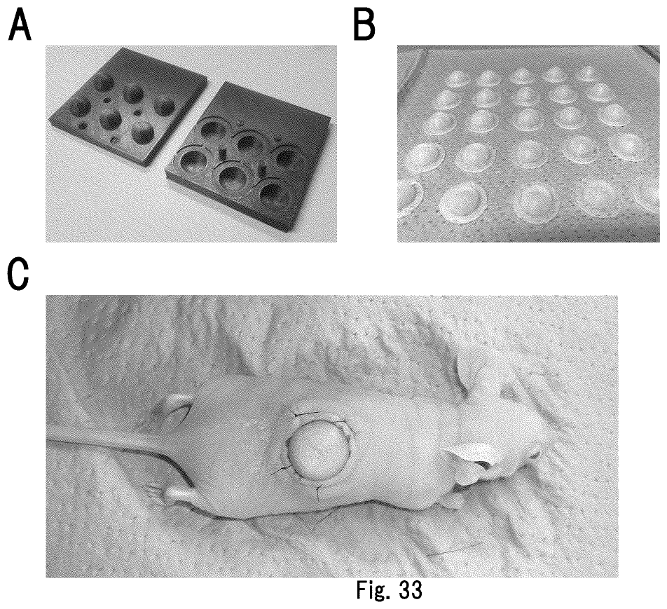

As the immunodeficient animals, BALB/cAJcl-nu/nu (from Central Institute for Experimental Animals) mice were used. As the silicone chamber, a chamber that could be attached to an animal according to a method described in Reference Literature 3 (Lichti U, Anders J, Yuspa S H, “Isolation and short-term culture of primary keratinocytes, hair follicle populations and dermal cells from newborn mice and keratinocytes from adult mice for in vitro analysis and for grafting to immunodeficient mice”, “Nat Protocol”, 2008, 3 (5), p 799-810) was prepared by pouring a molding silicone (HTV-4000, Engraving Japan Corporation) into a mold with an inner diameter of 10 mm, a brim width of 3 mm, and a thickness of 0.5 mm made by a 3D printer. FIG. 33(A) presents the mold of the silicone chamber, and FIG. 33(B) presents the prepared silicone chamber. A skin of a mouse back was excised in a circular shape, and then the silicone chamber was inserted and fixed thereto by suture with nylon thread. FIG. 33(C) presents a mouse with a silicone chamber sewn onto its back.

### Example 2

2-1 Induction of Induced Skin Appendage-Inducible Mesenchymal Cell from Primary Cultured Adult Mouse Adipose-Derived Mesenchymal Cell

Retroviruses that expressed ETV1 gene, FOXD1 gene, PRDM1 gene, SHH gene, and LEF1 gene for transfecting genes into adult mouse adipose-derived mesenchymal cells under a culture condition were prepared.

The retrovirus plasmid was prepared by: preparing a cDNA from Gateway human entry clone purchased from NBRC (Biological Resource Center, National Institute of Technology and Evaluation), BJ fibroblast (ATCC), and mRNA collected from human fibroblasts (Takara Bio Inc.); and using the cDNA as a template, subcloning a coding sequence amplified by PCR using In-Fusion® HD Cloning Kit (Clontech Laboratories, Inc.) into a PMXs retrovirus. Lipofectamine 2000 (Thermo Fisher Scientific) was used with packaging plasmids (pCMV-gagpol-PA, pCMV-VSVg) for transfection into a 293 FT cell (Thermo Fisher Scientific), and the cell supernatant after replacing the medium was used as a retrovirus solution.

A subcutaneous adipose specimen sampled from a waist-back site of an adult mouse (C57BL/6) was cut into small pieces and treated with 0.1% collagenase at 37° C. for 1 hour in order to collect cells. Then, the cells were seeded in a DMEM medium supplemented with 10% fetal bovine serum in order to collect primary cultured adult mouse adipose-derived mesenchymal cells. The mesenchymal cells were subcultured up to the second passage and then cultured in DMEM media (Polybrene 4 μg/ml) supplemented with 10% fetal bovine serum containing 10% SHH gene-expressing retrovirus solution and 10% LEF1 gene-expressing retrovirus solution in order to create first induced mesenchymal cells expressing SHH gene and LEF1 gene, and similarly cultured in DMEM media (Polybrene 4 μg/ml) supplemented with 10% fetal bovine serum containing 10% ETV1 gene-expressing retrovirus solution, 10% FOXD1 gene-expressing retrovirus solution, and 10% PRDM1 gene-expressing retrovirus solution, in order to create second induced mesenchymal cells expressing ETV1 gene, FOXD1 gene, and PRDM1 gene.

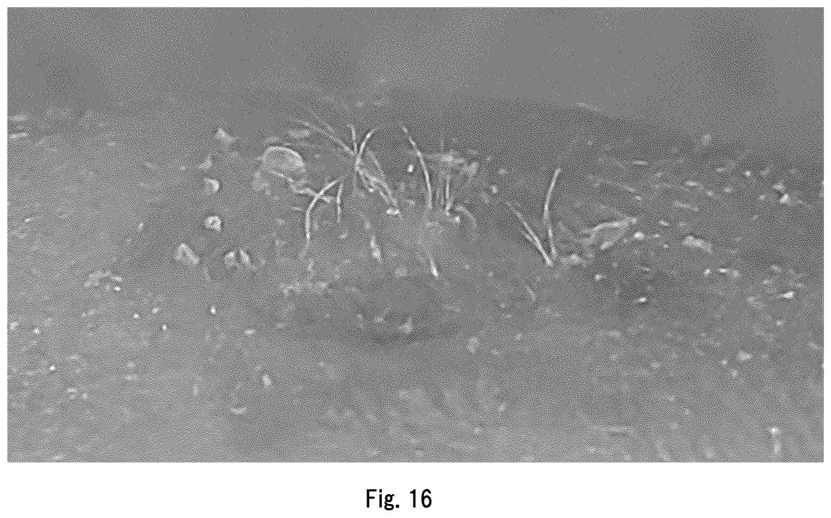

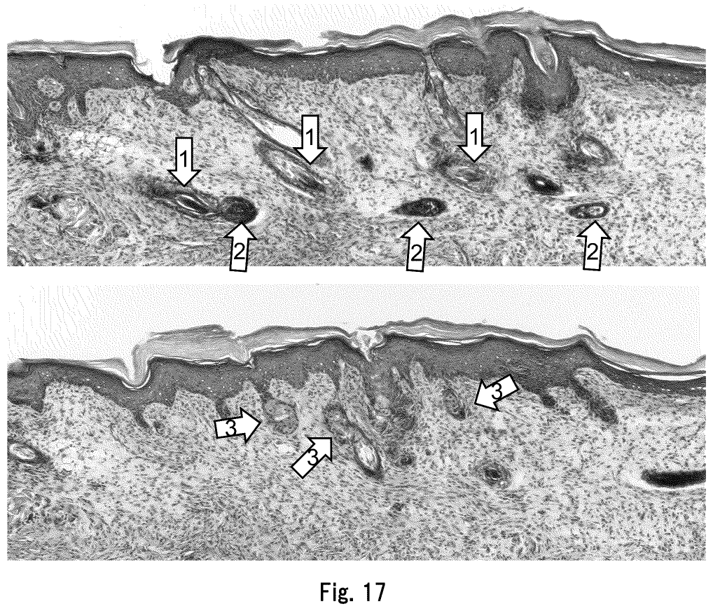

The induced epithelial cell obtained in Example 1-1 (by using the AAV that expresses DNP63A gene, GRHL2 gene, TFAP2A gene, c-MYC gene, and LEF1 gene) and the two types of induced mesenchymal cells obtained in Example 2-1 (the first induced mesenchymal cell obtained by using the retrovirus that expresses SHH gene and LEF1 gene, and the second induced mesenchymal cell obtained by using the retrovirus that expresses ETV1 gene, FOXD1 gene, and PRDM1 gene) were transplanted in combination into a chamber attached to a back of an immunodeficient animal. One week after the transplantation, a hole was formed on the top of the silicone chamber, and 2 weeks after the transplantation, the silicone chamber was removed, and observation was continued for 4 weeks after the transplantation. Hair growth as well as reconstruction and regeneration of skin appendages were achieved on the chamber site (FIG. 16). Regeneration of mature hair shafts, hair matrix tissue, and sebaceous gland tissue was observed at that site by a histological investigation (FIG. 17).

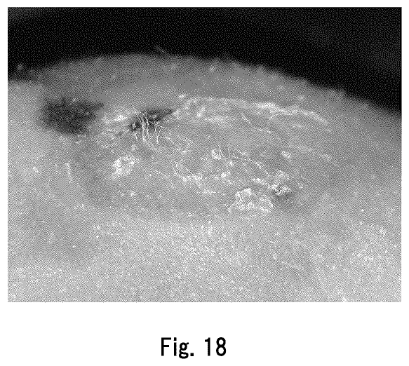

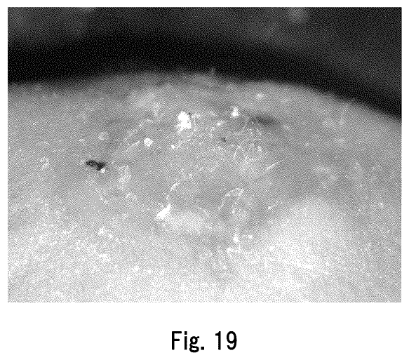

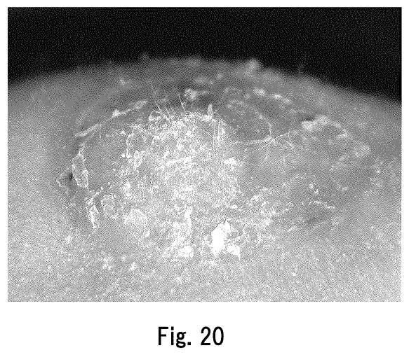

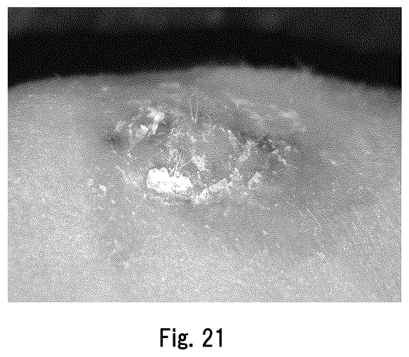

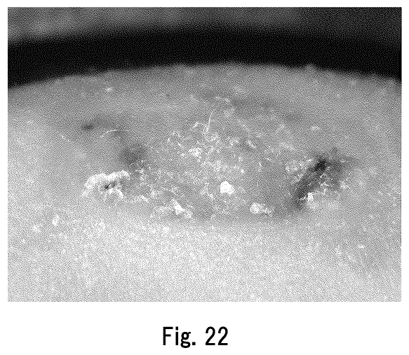

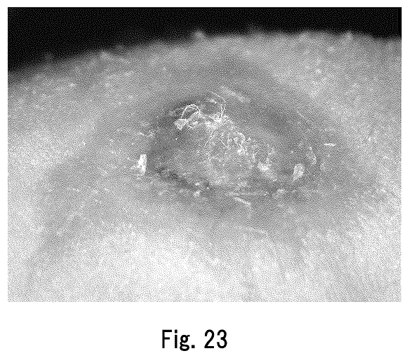

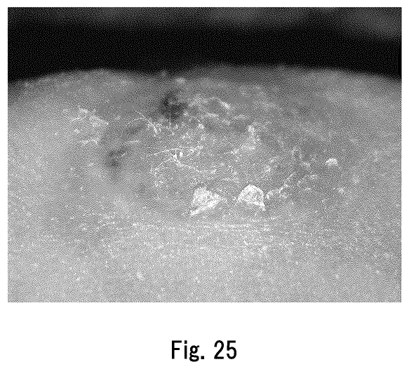

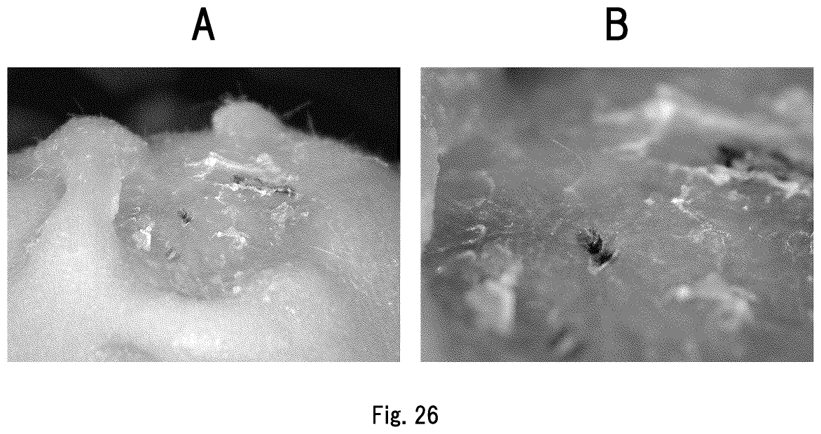

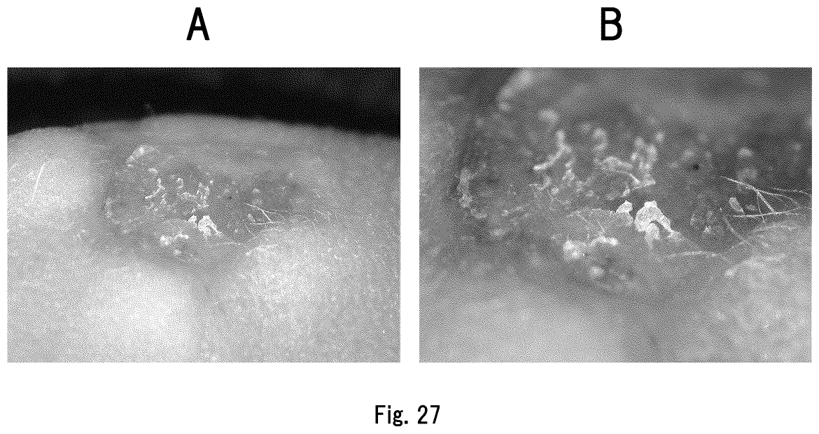

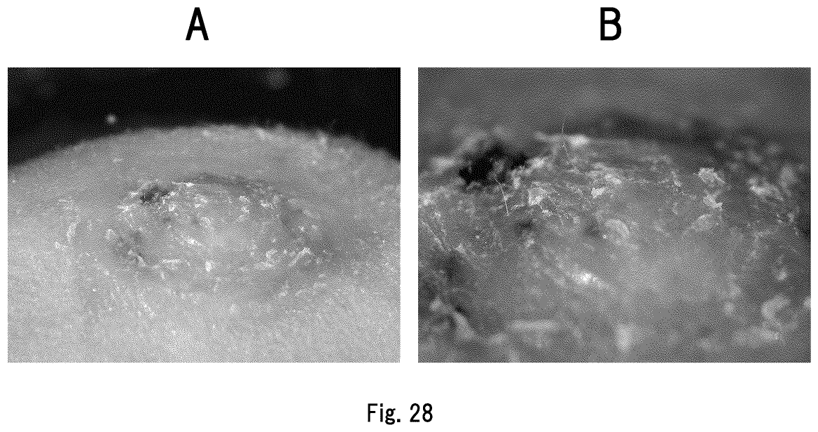

2-2 The induced epithelial cell induced by DNP63A gene, GRHL2 gene, TFAP2A gene, c-MYC gene, and LEF1 gene by the experimental method according to Example 1-1, as well as the first induced mesenchymal cell induced by SHH gene, and the second induced mesenchymal cell induced by ETV1 gene, FOXD1 gene, and PRDM1 gene by the experimental method according to Example 2-1, were transplanted in combination into a chamber attached to a back of an immunodeficient animal. One week after the transplantation, a hole was formed on the top of the silicone chamber, and 2 weeks after the transplantation, the silicone chamber was removed, and observation was continued for 4 weeks after the transplantation. Skin appendages were regenerated on the chamber site (FIG. 18).
2-3 The induced epithelial cell induced by DNP63A gene, GRHL2 gene, TFAP2A gene, c-MYC gene, and LEF1 gene by the experimental method according to Example 1-1, as well as the first induced mesenchymal cell induced by SHH gene and LEF1 gene, and the second induced mesenchymal cell induced by ETV1 gene and FOXD1 gene by the experimental method according to Example 2-1, were transplanted in combination into a chamber attached to a back of an immunodeficient animal. One week after the transplantation, a hole was formed on the top of the silicone chamber, and 2 weeks after the transplantation, the silicone chamber was removed, and observation was continued for 4 weeks after the transplantation. Skin appendages were regenerated on the chamber site (FIG. 19).
2-4 The induced epithelial cell induced by DNP63A gene, GRHL2 gene, TFAP2A gene, c-MYC gene, and LEF1 gene by the experimental method according to Example 1-1, as well as the first induced mesenchymal cell induced by SHH gene and LEF1 gene, and the second induced mesenchymal cell induced by FOXD1 gene and PRDM1 gene by the experimental method according to Example 2-1, were transplanted in combination into a chamber attached to a back of an immunodeficient animal. One week after the transplantation, a hole was formed on the top of the silicone chamber, and 2 weeks after the transplantation, the silicone chamber was removed, and observation was continued for 4 weeks after the transplantation. Skin appendages were regenerated on the chamber site (FIG. 20).
2-5 The induced epithelial cell induced by DNP63A gene, GRHL2 gene, TFAP2A gene, c-MYC gene, and LEF1 gene by the experimental method according to Example 1-1, as well as the first induced mesenchymal cell induced by SHH gene and LEF1 gene, and the second induced mesenchymal cell induced by ETV1 gene and PRDM1 gene by the experimental method according to Example 2-1, were transplanted in combination into a chamber attached to a back of an immunodeficient animal. One week after the transplantation, a hole was formed on the top of the silicone chamber, and 2 weeks after the transplantation, the silicone chamber was removed, and observation was continued for 4 weeks after the transplantation. Skin appendages were regenerated on the chamber site (FIG. 21).
2-6 The induced epithelial cell induced by DNP63A gene, GRHL2 gene, TFAP2A gene, c-MYC gene, and LEF1 gene by the experimental method according to Example 1-1, and the induced mesenchymal cell induced by ETV1 gene, FOXD1 gene, PRDM1 gene, and SHH gene by the experimental method according to Example 2-1, were transplanted in combination into a chamber attached to a back of an immunodeficient animal. One week after the transplantation, a hole was formed on the top of the silicone chamber, and 2 weeks after the transplantation, the silicone chamber was removed, and observation was continued for 4 weeks after the transplantation. Skin appendages were regenerated on the chamber site (FIG. 22).
2-7 The induced epithelial cell induced by DNP63A gene, GRHL2 gene, TFAP2A gene, c-MYC gene, and LEF1 gene by the experimental method according to Example 1-1, and the induced mesenchymal cell induced by ETV1 gene, FOXD1 gene, PRDM1 gene, SHH gene, and LEF1 gene by the experimental method according to Example 2-1, were transplanted in combination into a chamber attached to a back of an immunodeficient animal. One week after the transplantation, a hole was formed on the top of the silicone chamber, and 2 weeks after the transplantation, the silicone chamber was removed, and observation was continued for 4 weeks after the transplantation. Skin appendages were regenerated on the chamber site (FIG. 23).
2-8 The induced epithelial cell induced by DNP63A gene, GRHL2 gene, TFAP2A gene, c-MYC gene, and LEF1 gene by the experimental method according to Example 1-1, and the induced mesenchymal cell induced by SHH gene and LEF1 gene by the experimental method according to Example 2-1, were transplanted in combination into a chamber attached to a back of an immunodeficient animal. One week after the transplantation, a hole was formed on the top of the silicone chamber, and 2 weeks after the transplantation, the silicone chamber was removed, and observation was continued for 4 weeks after the transplantation. Skin appendages were regenerated on the chamber site (FIG. 24).
2-9 The induced epithelial cell induced by DNP63A gene, GRHL2 gene, TFAP2A gene, c-MYC gene, and LEF1 gene by the experimental method according to Example 1-1, as well as the first induced mesenchymal cell induced by SHH gene and LEF1 gene, and the second induced mesenchymal cell induced by PRDM1 gene by the experimental method according to Example 2-1, were transplanted in combination into a chamber attached to a back of an immunodeficient animal. One week after the transplantation, a hole was formed on the top of the silicone chamber, and 2 weeks after the transplantation, the silicone chamber was removed, and observation was continued for 4 weeks after the transplantation. Skin appendages were regenerated on the chamber site (FIG. 25).
2-10 The induced epithelial cell induced by DNP63A gene, GRHL2 gene, TFAP2A gene, c-MYC gene, and LEF1 gene by the experimental method according to Example 1-1, as well as the first induced mesenchymal cell induced by SHH gene, and the second induced mesenchymal cell induced by FOXD1 gene and PRDM1 gene by the experimental method according to Example 2-1, were transplanted in combination into a chamber attached to a back of an immunodeficient animal. One week after the transplantation, a hole was formed on the top of the silicone chamber, and 2 weeks after the transplantation, the silicone chamber was removed, and observation was continued for 4 weeks after the transplantation. Skin appendages were regenerated on the chamber site (FIG. 26).
2-11 The induced epithelial cell induced by DNP63A gene, GRHL2 gene, TFAP2A gene, c-MYC gene, and LEF1 gene by the experimental method according to Example 1-1, as well as the first induced mesenchymal cell induced by SHH gene, and the second induced mesenchymal cell induced by PRDM1 gene by the experimental method according to Example 2-1, were transplanted in combination into a chamber attached to a back of an immunodeficient animal. One week after the transplantation, a hole was formed on the top of the silicone chamber, and 2 weeks after the transplantation, the silicone chamber was removed, and observation was continued for 4 weeks after the transplantation. Skin appendages were regenerated on the chamber site (FIG. 27).
2-12 The induced epithelial cell induced by DNP63A gene, GRHL2 gene, TFAP2A gene, c-MYC gene, and LEF1 gene by the experimental method according to Example 1-1, as well as the first induced mesenchymal cell induced by SHH gene, and the second induced mesenchymal cell induced by FOXD1 gene by the experimental method according to Example 2-1, were transplanted in combination into a chamber attached to a back of an immunodeficient animal. One week after the transplantation, a hole was formed on the top of the silicone chamber, and 2 weeks after the transplantation, the silicone chamber was removed, and observation was continued for 4 weeks after the transplantation. Skin appendages were regenerated on the chamber site (FIG. 28).
2-13 The induced epithelial cell induced by DNP63A gene, GRHL2 gene, TFAP2A gene, c-MYC gene, and LEF1 gene by the experimental method according to Example 1-1, and the induced mesenchymal cell induced by SHH gene by the experimental method according to Example 2-1, were transplanted in combination into a chamber attached to a back of an immunodeficient animal. One week after the transplantation, a hole was formed on the top of the silicone chamber, and 2 weeks after the transplantation, the silicone chamber was removed, and observation was continued for 4 weeks after the transplantation. Skin appendages were regenerated on the chamber site (FIG. 29).
2-14 The induced epithelial cell induced by DNP63A gene, GRHL2 gene, TFAP2A gene, c-MYC gene, and LEF1 gene by the experimental method according to Example 1-1, and the induced mesenchymal cell induced by ETV1 gene, FOXD1 gene, and PRDM1 gene by the experimental method according to Example 2-1, were transplanted in combination into a chamber attached to a back of an immunodeficient animal. One week after the transplantation, a hole was formed on the top of the silicone chamber, and 2 weeks after the transplantation, the silicone chamber was removed, and observation was continued for 4 to 5 weeks after the transplantation. FIG. 30(A) is a photograph of an appearance on day 28 (D28), and (B) is a cross-sectional photograph of hematoxylin eosin-stained (HE) tissue (the upper photograph is an entire image, and the lower three photographs are partially enlarged images). Although hair growth is not observed in the appearance photograph, hair follicle-like structures and mature hair shafts were observed by a histological investigation on a central subcutaneous region of the cell transplantation site where hairs were not normally observed. The induced mesenchymal cells induced by ETV1 gene, FOXD1 gene, and PRDM1 gene produced in Example 2 were transplanted as the second induced mesenchymal cells together with the first induced mesenchymal cells in Examples 2-1 and 2-2. However, it was confirmed that the induced mesenchymal cells could induce hair follicle-like structures and matured hair shafts even when transplanted alone. The induced mesenchymal cells having such an inducibility can be transplanted into e.g., a site that physiologically express a high level of SHH or LEF1 to regenerate skin appendages. Furthermore, as can be seen from the result of Example 2, skin and skin appendage-like tissues can be produced in vitro by transgenes including ETV1 gene, FOXD1 gene, and PRDM1 gene, and can be used for transplantation and other applications. Also, the induced mesenchymal cells can be used as a part of a skin appendage-inducible cell population by transplantation in combination with cells that physiologically express a high level of SHH.

### Example 3

Regeneration of Skin Appendage by Other Induced Skin Appendage-Inducible Epithelial and Mesenchymal Cells Induced from Primary Cultured Adult Mouse Adipose-Derived Mesenchymal Cell

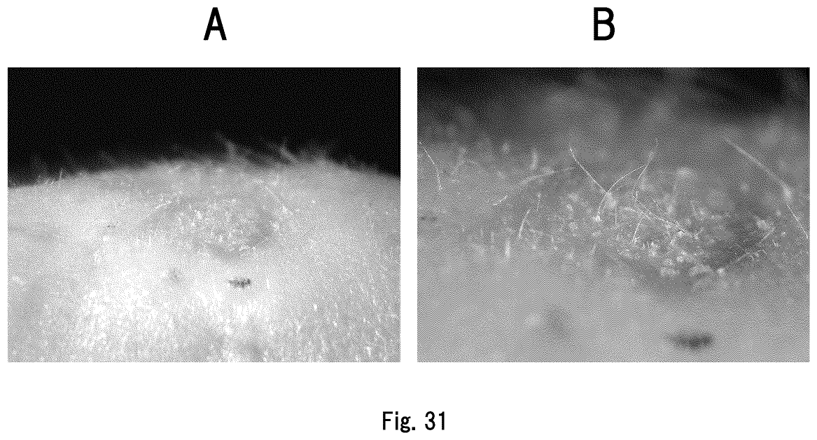

In Example 3, four genes: DNP63A gene, GRHL2 gene, TFAP2A gene, and LEF1 gene were adopted as transgenes for induced epithelial cells. The induced epithelial cells induced by DNP63A gene, GRHL2 gene, TFAP2A gene, and LEF1 gene by the experimental method according to Example 1-1, the first induced mesenchymal cells induced by SHH gene and LEF1 gene, and the second induced mesenchymal cells induced by ETV1 gene, FOXD1 gene, and PRDM1 genes, were transplanted in combination into a chamber attached to a back of an immunocompromised animal. One week after the transplantation, a hole was formed on the top of the silicone chamber, and 2 weeks after the transplantation, the silicone chamber was removed, and observation was continued for 4 weeks after the transplantation (FIG. 31). Skin appendages were regenerated on the chamber site (FIG. 31)

As can be seen from Examples 1 and 3, the induced skin appendage-inducible epithelial cells could be produced by transducing five genes: DNP63A gene, GRHL2 gene, TFAP2A gene, c-MYC gene, and LEF1 gene, or four genes: DNP63A gene, GRHL2 gene, TFAP2A gene, and LEF1 gene, into skin appendage-uninducible somatic cells. Since induced epithelial cells could not be obtained in Comparative Examples 1 to 3, it was turned out that LEF1 gene transduction was required for producing skin appendage-inducible epithelial cells. As the transgene to be transduced in combination with LEF1 gene, it is preferable to transduce one or more genes selected from DNP63A gene, GRHL2 gene, TFAP2A gene, and c-MYC gene.

Also, as can be seen from Example 2-13, the induced skin appendage-inducible mesenchymal cells could be produced by transducing SHH gene into skin appendage-uninducible somatic cells. In addition, as can be seen from Example 2-8, the induced skin appendage-inducible mesenchymal cells could be produced by transducing SHH gene and LEF1 gene into skin appendage-uninducible somatic cells. Furthermore, as can be seen from Example 2-14, the induced skin appendage-inducible mesenchymal cells could be produced by transducing ETV1 gene, PRDM1 gene, and FOXD1 gene into skin appendage-uninducible somatic cells. It is suggested from FIG. 32 that these cells can be simultaneously transplanted to acquire a higher skin appendage-inducing ability. In particular, it is suggested that cells transfected with a part of ETV1 gene, PRDM1 gene, and FOXD1 gene, and cells transfected with SHH gene alone or with SHH gene and LEF1 gene can be simultaneously transplanted to produce induced mesenchymal cells having a higher skin appendage-inducing ability.

1. A method for producing a skin appendage-inducible cell, including a step of transfecting a transgene containing at least one gene that is relatively strongly expressed in a skin appendage-inducible cell into a skin appendage-uninducible somatic cell.

2. The method for producing a skin appendage-inducible cell according to claim 1, including a step of transfecting a transgene containing:
(1) LEF1 gene; and
(2) one, two, or three genes selected from DNP63A gene, GRHL2 gene, and TFAP2A gene into a skin appendage-uninducible somatic cell.

3. The method for producing a skin appendage-inducible cell according to claim 2, including a step of transfecting a transgene containing four genes: LEF1 gene, DNP63A gene, GRHL2 gene, and TFAP2A gene, into a skin appendage-uninducible somatic cell.

4. The method for producing a skin appendage-inducible cell according to claim 1, including a step of transfecting a transgene containing:
(1) LEF1 gene; and
(2) one, two, three, or four genes selected from DNP63A gene, GRHL2 gene, TFAP2A gene, and at least one MYC family genes into a skin appendage-uninducible somatic cell.

5. The method for producing a skin appendage-inducible cell according to claim 4, including a step of transfecting a transgene containing at least 5 genes: LEF1 gene, DNP63A gene, GRHL2 gene, TFAP2A gene, and at least one MYC family genes, into a skin appendage-uninducible somatic cell.

6. The method for producing the skin appendage-inducible cell according to claim 4, wherein the transgene includes cMYC gene as the MYC family gene.

7. The method for producing a skin appendage-inducible cell according to claim 1, including a step of transfecting a transgene containing one or more genes selected from SHH gene, ETV1 gene, FOXD1 gene, PRDM1 gene, and LEF1 gene, into a skin appendage-uninducible somatic cell.

8. The method for producing a skin appendage-inducible cell according to claim 1, including a step of transfecting a transgene containing SHH gene into a skin appendage-uninducible somatic cell.

9. The method for producing a skin appendage-inducible cell according to claim 1, including a step of transfecting a transgene containing SHH gene and LEF1 gene into a skin appendage-uninducible somatic cell.

10. The method for producing a skin appendage-inducible cell according to claim 1, including a step of transfecting a transgene containing one, two, or three genes selected from ETV1 gene, PRDM1 gene, and FOXD1 gene into a skin appendage-uninducible somatic cell.

11. The method for producing a skin appendage-inducible cell according to claim 1, including a step of transfecting a transgene containing:
(1) SHH gene; and
(2) one or more genes selected from ETV1 gene, PRDM1 gene, FOXD1 gene, and LEF1 gene into a skin appendage-uninducible somatic cell.

12. The method for producing a skin appendage-inducible cell according to claim 1, including a step of transfecting a transgene containing:
(1) ETV1 gene and PRDM1 gene;
(2) FOXD1 gene and PRDM1 gene; or
(3) ETV1 gene, PRDM1 gene, and FOXD1 gene into a skin appendage-uninducible somatic cell.

13. The method for producing the skin appendage-inducible cell according to claim 7, wherein a plurality of cells having different transgenes are prepared for transfecting multiple types of the genes.

14. The method for producing the skin appendage-inducible cell according to claim 1, wherein the somatic cell is derived from human.

15. The method for producing the skin appendage-inducible cell according to claim 1, wherein the somatic cell is a skin fibroblast, a subcutaneous adipose tissue-derived stromal cell (subcutaneous adipocyte), an embryonic fibroblast, an adipocyte, a muscle cell, an osteoblast, a cartilage cell, a monocyte in circulating blood, or somatic cells differentiated from an ES cell, a mesenchymal stem cell, or the like.

16. A skin appendage-inducible cell produced by the production method according to claim 1.

17. A cell preparation containing the skin appendage-inducible cell according to claim 16.

18. (canceled)

19. (canceled)

20. The cell preparation according to claim 17, containing a scaffold material.

21. The cell preparation according to claim 20, wherein the scaffold material is collagen.

22. The cell preparation according to claim 17, wherein the cell preparation is for a purpose of regenerating skin tissues or hairs.

23. The cell preparation according to claim 17, wherein the cell preparation is a sheet-like structure or a cell aggregate having a three-dimensional structure.

24. A non-human mammal having a formed skin appendage, the non-human mammal being produced by administering the skin appendage-inducible cell according to claim 16 into the non-human mammal to form the skin appendage from the skin appendage-inducible cell in a body of the mammal.

25. A method for determining a drug efficacy of a test substance, including a step of administering the test substance to the non-human mammal according to claim 24 to determine the drug efficacy of the test substance on skin and/or a skin appendage tissue.

26. A method for determining an influence of an external factor, including a step of loading the non-human mammal according to claim 24 with a stress such as an anticancer drug and a radiation to determine a stress on skin and/or a skin appendage tissue.

27. A skin appendage-inducible cell preparation composition containing a transgene used in the production method according to claim 1.

28. The cell preparation composition according to claim 27, containing the transgene in a form transfectable into a somatic cell.

29. A skin tissue and skin appendage-like tissue produced by culturing the skin appendage-inducible cell according to claim 16.

30. A method for analyzing a drug efficacy of a test substance, including a step of administering the test substance to the skin tissue and/or skin appendage-like tissue according to claim 29 to analyze the drug efficacy of the test substance on the skin tissue and/or skin appendage-like tissue.

31. A method for analyzing an influence of an external factor, including a step of loading the skin tissue and/or skin appendage-like tissue according to claim 29 with a stress such as an anticancer drug or a radiation to analyze a stress on the skin tissue and/or skin appendage-like tissue.

32. A kit of a vector specialized for transfecting the gene according to claim 1 into a somatic cell.

33. A method for evaluating a skin appendage-inducing ability of a cell, using the skin appendage-inducible cell according to claim 16.

34. (canceled)

35. (canceled)

36. (canceled)

37. (canceled)

38. The kit of the vector according to claim 32, wherein the vector is a viral vector.

39. A cell preparation containing a skin appendage-inducible cell produced by the production method according to claim 2 and a skin appendage-inducible mesenchymal cell.

40. The cell preparation according to claim 39, wherein the skin appendage-inducible mesenchymal cell is a skin appendage-inducible cell produced by the production method according to claim 7.

41. A cell preparation containing a skin appendage-inducible cell produced by the production method according to claim 7 and a skin appendage-inducible epithelial cell.

42. The cell preparation according to claim 41, wherein the skin appendage-inducible epithelial cell is a skin appendage-inducible cell produced by the production method according to claim 2.

43. A non-human mammal having a formed skin appendage, the non-human mammal being produced by administering the cell preparation according to claim 17 into the non-human mammal to form the skin appendage from the skin appendage-inducible cell in a body of the mammal.
